(19) United States
(12) Patent Application Publication
(10) Pub. No.: US 2019/0092800 A1
(43) Pub. Date: Mar. 28, 2019

(54) LIQUID CHROMATOGRAPHIC SEPARATION OF CARBOHYDRATE TAUTOMERS

(71) Applicant: Waters Technologies Corporation, Milford, MA (US)

(72) Inventors: Charles H. Phoebe, Jr., Uxbridge, MA (US); Aaron D. Phoebe, Uxbridge, MA (US)

(21) Appl. No.: 16/143,266

(22) Filed: Sep. 26, 2018

Related U.S. Application Data
(60) Provisional application No. 62/563,358, filed on Sep. 26, 2017.

(51) Int. Cl. C07H 1/06; G01N 30/74; B01J 20/281

(52) U.S. Cl. CPC C07H 1/06; G01N 2030/027; G01N 30/482; G01N 30/74

(57) ABSTRACT

The present invention provides a novel, simple and reliable method for the separation of carbohydrate tautomers. The method comprises steps of chromatographically separating a sample using a chromatographic device. The method can be used to separate mono- and disaccharides tautomeric species including arabinose, xylose, fructose, mannose, galactose, glucose, lactose, and maltose.

### RELATED APPLICATIONS

This application claims the benefit of U.S. Provisional Application No. 62/563,358, filed Sep. 26, 2017, the entire disclosure of which is incorporated by reference.

### BACKGROUND OF THE INVENTION

The exact molecular structures of carbohydrates in solution is an area of research that has received considerable attention due to their relevance to biological systems. The changes in optical rotation of carbohydrates in solution, referred to as mutarotation, has been studied since the mid-19th century. Since then, numerous methods have been used to measure and to characterize this observation including spectroscopic (polarimetric and nuclear magnetic resonance (NMR)) and gas chromatographic (GC) techniques. The conclusion from these studies suggested the occurrence of mutarotation as a result of the interconversion among distinct chemical cyclic forms, refer to as tautomers, for carbohydrates in solution.

The tautomers can be identified individually by NMR spectroscopic analysis, which requires the deconvolution of the overlapping spectra, or by GC, which requires derivatization prior to analysis. Liquid chromatographic (LC) analysis has an advantage over gas chromatographic analysis because no derivatization is required prior to analysis; however, no effective LC method has been reported in separating carbohydrate tautomers because of the poor resolution, if there is any, of different tautomeric species.

The current invention provides a novel LC method in separating carbohydrate tautomeric species, including but not limited to mono- and di-saccahrides, e.g. arabinose, xylose, fructose, mannose, galactose, glucose, lactose, maltose, sorbose, ribose, psicose, trehalose, raffinose, altrose, tagatose, lyxose, turanose, allose, gulose, palatinose, and their mixtures.

### SUMMARY OF THE INVENTION

The present invention provides a novel, simple, reliable and sensitive liquid chromatographic method for separating and quantitating carbohydrate tautomeric species, for example, arabinose, xylose, fructose, mannose, galactose, glucose, lactose, maltose, sorbose, ribose, psicose, trehalose, raffinose, altrose, tagatose, lyxose, turanose, allose, gulose, palatinose, or their mixtures.

Moreover, the invention provides an option of further detection of separated species (from liquid chromatographic device) by a mass spectrometer, charged aerosol, polarimeter, pulsed amperometric electrochemical, condensation nucleation light scattering or NMR detectors.

In one embodiment, the invention provides a method of chromatographically separating carbohydrate tautomeric species using a chromatographic device with a column packed with chromatographic stationary phase represented by Formula 1

[X](W)a(Q)b(T)c Formula 1

wherein: X is a high purity chromatographic core composition having a surface comprising a silica core material, metal oxide core material, an inorganic-organic hybrid material or a group of block copolymers thereof; W is absent and/or includes hydrogen and/or includes hydroxyl on the surface of X; Q is a functional group that minimizes retention variation over time (drift) under chromatographic conditions utilizing low water concentrations; T comprises one or more hydrophilic, polar, ionizable, and/or charged functional groups that chromatographically interact with the analyte; and b and c are positive numbers, 0.05<(b/c)<100, and a>0.

In another embodiment, the invention provides a method of chromatographically separating carbohydrate tautomeric species using a chromatographic device with a column packed with chromatographic stationary phase represented by Formula 1; wherein: X is a chromatographic core material comprising a silica based, metal oxide based, or inorganic-organic hybrid based core surface; W is absent and/or includes hydrogen and/or includes hydroxyl on the surface of X; Q comprises one or more functional groups that essentially prevent chromatographic interaction between an analyte, and X and W, wherein a first fraction of Q is bound to X and a section fraction of Q is polymerized; T comprises one or more hydrophilic, polar, ionizable, and/or charged functional groups that chromatographically interact with the analyte, wherein a third fraction of T is bound to Q, a fourth fraction of T is bound to X, and a fourth fraction of T is polymerized; and b and c are positive numbers, 0.05<(b/c)<100, and a>0.

In yet another embodiment, the invention provides a method of chromatographically separating carbohydrate tautomeric species using a chromatographic device with an evaporative light scattering (ELS) detector, and/or a refractive index (RI) detector.

In another embodiment, the invention provides a method of chromatographically separating mono- and disaccharides tautomeric species and their mixtures.

In another embodiment, the invention provides a method of chromatographically separating tautomeric species of arabinose, xylose, fructose, mannose, galactose, sucrose, glucose, lactose, maltose, sorbose, ribose, psicose, trehalose, raffinose, altrose, tagatose, lyxose, turanose, allose, gulose, palatinose, or their mixtures.

In yet another embodiment, the invention provides a method of monitoring mutarotation of D-(+)-glucose tautomers in water.

In another embodiment, the invention provides a method of chromatographically separating fructose tautomers including β-D-fructopyranose, β-D-fructofuranose, α-D-fructopyranose, and α-D-fructofuranose.

In another embodiment, the invention provides a method of chromatographically separating glucose tautomers of β-D-glucopyranose and α-D-glucopyranose.

### BRIEF DESCRIPTION OF THE DRAWINGS

The above and other features of the present invention will now be described in detail with reference to certain exemplary embodiments thereof illustrated the accompanying drawings which are given herein below by way of illustration only, and thus are not limitative of the present invention, and wherein:

FIG. 1 illustrates overlaid chromatograms where each carbohydrate is a single peak. Even though some of the peaks are broad, no resolution of the individual tautomers are present. (ACQUITY UPC2™ Torus DEA (diethylamine) column at a column temperature of 20° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 2 illustrates overlaid chromatograms where each carbohydrate is a single peak. Even though some of the peaks are broad, in general no resolution of the individual tautomers are present. The exception to this is that a small peak is appearing in front of Peak 5 (galactose). (ACQUITY UPC2™ BEH 2-EP (2-ethylpyridine) column at a column temperature of 20° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 3 illustrates overlaid chromatograms where each carbohydrate is a single peak. Note that the temperature is significantly higher in figure three. This prevents the resolution of the tautomers. If the temperature is dropped by 10° C. (Figure Four), to 40, the peaks start to show significant peak broadening (peaks 8 and 9) and peak splitting (peaks 5 and 6) due to the tautomeric resolution. (ACQUITY UPC2™ Torus 2-PIC (2-picolylamine) column at a column temperature of 50° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 4 illustrates broadened peaks and some peak splitting due to the resolution of the tautomers, the overall separation of the tautomers is poor. (ACQUITY UPC2™ Torus 2-PIC (2-picolylamine) column at a column temperature of 40° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 5 illustrates a column commercialized for the analysis of mono- and di-saccharides. The chromatographic conditions show single sharp peak for all compounds. There is no indication of tautomeric resolution. Carbohydrate peak assignment are as follows: 1. Arabinose, 2. Xylose, 3. Fructose, 4. Mannose, 5. Galactose, 6. Glucose, 7. Sucrose, 8. Lactose and 9. Maltose.

FIG. 6 illustrates a commercialized column used under exploratory conditions for the analysis of mono-and di-saccharides. The peaks are broad especially for peaks 7-9, but no indication of tautomeric resolution is present. (FLARE HILIC column at a column temperature of 50° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 7 illustrates multiple peaks demonstrating tautomeric resolution for all reducing mono- and disaccharides. The non-reducing disaccharide, sucrose (peak 7) is present as a single peak. (ACQUITY UPC2™ Torus Diol column at a column temperature of 20° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid. Carbohydrate peak assignment as follows: 1. Arabinose, 2. Xylose, 3. Fructose, 4. Mannose, 5. Galactose, 6. Glucose, 7. Sucrose, 8. Lactose and 9. Maltose.)

FIG. 8 illustrates a subset of the mono- and disaccharides (peaks 1, 3, 5 and 8) in relation to the single peak 7 for sucrose. Sharp highly resolved peaks for the tautomeric species are observed. (Same condition as FIG. 7.)

FIG. 9 illustrates a subset of the mono- and disaccharides (peaks 2, 3, 4, 6 and 9) in relation to the single peak 7 for sucrose. Sharp highly resolved peaks for the tautomeric species are observed. (Same condition as FIG. 7.)

FIG. 10 a. illustrates the separation of L-(+)-arabinose tautomers, same condition as FIG. 7 (column temperature of 20° C.); b. illustrates the separation of L-(+)-arabinose tautomers at a column temperature of 20° C., 30° C., 40° C. and 50° C. (other conditions the same); c. illustrates the separation of L-(+)-arabinose tautomers at different % of acetonitrile in water (column temperature of 20° C., other conditions the same).

FIG. 11 a illustrates the separation D-(+)-xylose tautomers, same condition as FIG. 7 (column temperature of 20° C.); b. illustrates the separation of D-(+)-xylose tautomers at a column temperature of 20° C., 30° C., 40° C. and 50° C. (other conditions the same); c. illustrates the separation of D-(+)-xylose tautomers at different % of acetonitrile in water (column temperature of 20° C., other conditions the same).

FIG. 12 a. illustrates the separation of D-(−)-fructose tautomers, same condition as FIG. 7 (column temperature of 20° C.); b. illustrates the separation of D-(−)-fructose tautomers at a column temperature of 20° C., 30° C., 40° C. and 50° C. (other conditions the same); c. illustrates the separation of D-(−)-fructose tautomers at different % of acetonitrile in water (column temperature of 20° C., other conditions the same).

FIG. 13 a. illustrates the separation of D-(+)-glucose tautomers, same condition as FIG. 7 (column temperature of 20° C.); b. illustrates the separation of D-(+)-glucose tautomers at a column temperature of 20° C., 30° C., 40° C. and 50° C. (other conditions the same); c. illustrates the separation of D-(+)-glucose tautomers at different % of acetonitrile in water (column temperature of 20° C., other conditions the same).

FIG. 14 a. illustrates the separation of D-(+)-mannose tautomers, same condition as FIG. 7 (column temperature of 20° C.); b. illustrates the separation of D-(+)-mannose tautomers at a column temperature of 20° C., 30° C., 40° C. and 50° C. (other conditions the same); c. illustrates the separation of D-(+)-mannose tautomers at different % of acetonitrile in water (column temperature of 20° C., other conditions the same).

FIG. 15 a. illustrates the separation of D-(+)-galactose tautomers, same condition as FIG. 7 (column temperature of 20° C.); b. illustrates the separation of D-(+)-galactose tautomers at a column temperature of 20° C., 30° C., 40° C. and 50° C. (other conditions the same); c. illustrates the separation of D-(+)-galactose tautomers at different % of acetonitrile in water (column temperature of 20° C., other conditions the same).

FIG. 16 a. illustrates sucrose analysis, same condition as FIG. 7 (column temperature of 20° C.); b. illustrates sucrose analysis at a column temperature of 20° C., 30° C., 40° C. and 50° C. (other conditions the same); c. illustrates sucrose analysis at different % of acetonitrile in water (column temperature of 20° C., other conditions the same). Because the disaccharide is not a reducing sugar, there are no tautomers to resolve and there is only a single peak.

FIG. 17 a. illustrates the separation of D-(+)-maltose (disaccharide) tautomers, same condition as FIG. 7 (column temperature of 20° C.); b. illustrates the separation of D-(+)-maltose tautomers at a column temperature of 20° C., 30° C., 40° C. and 50° C. (other conditions the same); c. illustrates the separation of D-(+)-maltose tautomers at different % of acetonitrile in water (column temperature of 20° C., other conditions the same).

FIG. 18 a. illustrates the separation of lactose (disaccharide) tautomers, same condition as FIG. 7 (column temperature of 20° C.); b. illustrates the separation of lactose tautomers at a column temperature of 20° C., 30° C., 40° C. and 50° C. (other conditions the same); c. illustrates the separation of lactose tautomers at different % of acetonitrile in water (column temperature of 20° C., other conditions the same).

FIG. 19 illustrates the separation of D-(−)-ribose tautomers. (Same condition as FIG. 7.)

FIG. 20 illustrates the separation of L-(−)-lyxose tautomers. (Same condition as FIG. 7.)

FIG. 21 illustrates the separation of D-allose tautomers. (Same condition as FIG. 7.)

FIG. 22 illustrates the separation of D-altrose tautomers. (Same condition as FIG. 7.)

FIG. 23 illustrates the separation of L-(+)-gulose tautomers. (Same condition as FIG. 7.)

FIG. 24 illustrates the separation of D-psicose tautomers. (Same condition as FIG. 7.)

FIG. 25 illustrates the separation of D-(+)-sorbose tautomers. (Same condition as FIG. 7.)

FIG. 26 illustrates the separation of D-(−)-tagatose tautomers. (Same condition as FIG. 7.)

FIG. 27 illustrates the separation of D-(+)-turanose tautomers. (Same condition as FIG. 7.)

FIG. 28 illustrates the separation of palatinose tautomers. (Same condition as FIG. 7.)

FIG. 29 illustrates the mutarotation of D-(+)-glucose tautomers in water (same condition as FIG. 7); a. injection made every 5 minutes for a total of 40 injections; b. injections 1, 10 and 40 (insert % of gluclose mutarotation in water as a function of injection/time).

FIG. 30 illustrates that increasing the column temperature (a. 40° C.; b. 50° C.; c. 60° C.) for Shodex SZ5532 column can collapse the tautomers (Shodex SZ5532 column (150 mm×4.6 mm i.d., particle size 6 μm), eluent consisting of acetonitrile and water (70/30) at a flow rate of 0.6 mL/min with ELS detection at a nitrogen pressure of 30 psi and a drift tube temperature of 55° C.

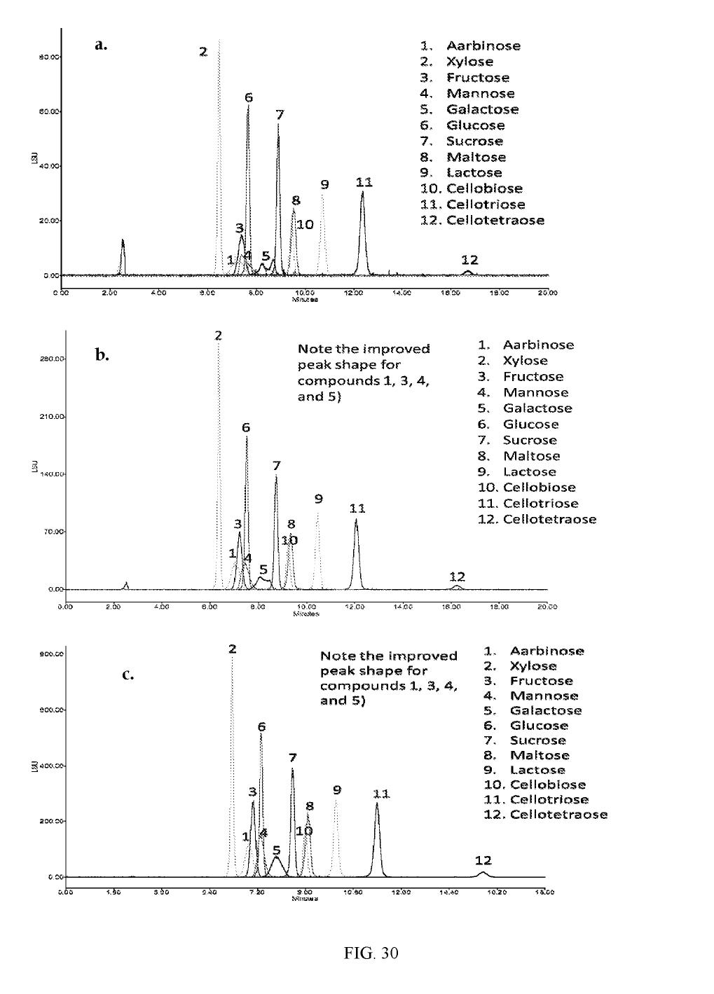

FIG. 31 illustrates the separation of L-(+)-arabinose tautomers using Glycan Amide Column (ACQUITY UPLC Glycan BEH Amide column(50 mm×2.1 mm i.d., particle size 1.7 μm) at a column temperature of 20° C., eluent consisting of various concentrations of acetonitrile and water (86/14) at a flow rate of 0.2 mL/min with ELS detection at a nitrogen pressure of 30 psi and a drift tube temperature of 55° C.).

FIG. 32 illustrates the separation of D-(+)-xylose tautomers using Glycan Amide Column (same condition as FIG. 31).

FIG. 33 illustrates the separation of D-(−)-fructose tautomers using Glycan Amide Column (same condition as FIG. 31).

FIG. 34 illustrates the separation of D-(+)-glucose tautomers using Glycan Amide Column (same condition as FIG. 31).

FIG. 35 illustrates the separation of D-(+)-mannose tautomers using Glycan Amide Column (same condition as FIG. 31).

FIG. 36 illustrates the separation of D-(+)-galactose tautomers using Glycan Amide Column (same condition as FIG. 31).

FIG. 37 illustrates sucrose analysis using Glycan Amide Column (same condition as FIG. 31).

FIG. 38 illustrates the separation of D-(+)-maltose tautomers using Glycan Amide Column (same condition as FIG. 31).

FIG. 39 illustrates the separation of lactose tautomers using Glycan Amide Column (same condition as FIG. 31).

### DETAILED DESCRIPTION OF THE INVENTION

The present invention provides a method for separation of carbohydrate tautomers from their mixtures using a liquid chromatographic device. The method can be used to separate mono-and disaccharides tautomeric species including arabinose, xylose, fructose, mannose, galactose, glucose, lactose, and maltose. The present invention will be more illustrated by following detailed description.

### Definitions

“High Purity” or “high purity chromatographic material” includes a material which is prepared form high purity precursors. In certain aspects, high purity materials have reduced metal contamination and/or non-diminished chromatographic properties including, but not limited to, the acidity of surface silanols and the heterogeneity of the surface.

“Chromatographic surface” includes a surface which provides for chromatographic separation of a sample. In certain aspects, the chromatographic surface is porous. In some aspects, a chromatographic surface can be the surface of a particle, a superficially porous material or a monolith. In certain aspects, the chromatographic surface is composed of the surface of one or more particles, superficially porous materials or monoliths used in combination during a chromatographic separation. In certain other aspects, the chromatographic surface is non-porous.

“Ionizable modifier” includes a functional group which bears an electron donating or electron withdrawing group. In certain aspects, the ionizable modifier contains one or more carboxylic acid groups, amino groups, imido groups, amido groups, pyridyl groups, imidazolyl groups, ureido groups, thionyl-ureido groups or aminosilane groups, or a combination thereof. In other aspects, the ionizable modifier contains a group bearing a nitrogen or phosphorous atom having a free electron lone pair. In certain aspects, the ionizable modifier is covalently attached to the material surface and has an ionizable group. In some instances it is attached to the chromatographic material by chemical modification of a surface hybrid group.

The term “hydrophilic” describes having an affinity for, attracting, adsorbing or absorbing water.

The term “hydrophobic” describes lacking an affinity for, repelling, or failing to adsorb or absorb water.

The term “ion-exchange functional group” is intended to include a group where the counter-ion is partially free and can readily be exchanged for other ions of the same sign.

“Hydrophobic surface group” includes a surface group on the chromatographic surface which exhibits hydrophobicity. In certain aspects, a hydrophobic group can be a carbon bonded phase such as a C4 to Cig bonded phase. In other aspects, a hydrophobic surface group can contain an embedded polar group such that the external portion of the hydrophobic surface maintains hydrophobicity. In some instances it is attached to the chromatographic material by chemical modification of a surface hybrid group. In other instances the hydrophobic group can be C4-C30, embedded polar, chiral, phenylalkyl, or pentafluorophenyl bonding and coatings.

“Chromatographic core” includes chromatographic materials, including but not limited to an organic material such as silica or a hybrid material, as defined herein, in the form of a particle, a monolith or another suitable structure which forms an internal portion of the materials of the invention. In certain aspects, the surface of the chromatographic core represents the chromatographic surface, as defined herein, or represents a material encased by a chromatographic surface, as defined herein. The chromatographic surface material can be disposed on or bonded to or annealed to the chromatographic core in such a way that a discrete or distinct transition is discernible or can be bound to the chromatographic core in such a way as to blend with the surface of the chromatographic core resulting in a gradation of materials and no discrete internal core surface. In certain embodiments, the chromatographic surface material can be the same or different from the material of the chromatographic core and can exhibit different physical or physiochemical properties from the chromatographic core, including, but not limited to, pore volume, surface area, average pore diameter, carbon content or hydrolytic pH stability.

“Hybrid,” including “hybrid inorganic/organic material,” includes inorganic-based structures wherein an organic functionality is integral to both the internal or “skeletal” inorganic structure as well as the hybrid material surface. The inorganic portion of the hybrid material can be, e.g., alumina, silica, titanium, cerium, or zirconium or oxides thereof, or ceramic material. “Hybrid” includes inorganic-based structures wherein an organic functionality is integral to both the internal or “skeletal” inorganic structure as well as the hybrid material surface. As noted above, exemplary hybrid materials are shown in U.S. Pat. Nos. 4,017,528, 6,528,167, 6,686,035 and 7,175,913, the contents of which are incorporated herein by reference in their entirety.

The term “alicyclic group” includes closed ring structures of three or more carbon atoms. Alicyclic groups include cycloparaffins or naphthenes which are saturated cyclic hydrocarbons, cycloolefins, which are unsaturated with two or more double bonds, and cycloacetylenes which have a triple bond. They do not include aromatic groups. Examples of cycloparaffins include cyclopropane, cyclohexane and cyclopentane. Examples of cycloolefins include cyclopentadiene and cyclooctatetraene. Alicyclic groups also include fused ring structures and substituted alicyclic groups such as alkyl substituted alicyclic groups. In the instance of the alicyclics such substituents can further comprise a lower alkyl, a lower alkenyl, a lower alkoxy, a lower alkylthio, a lower alkylamino, a lower alkylcarboxyl, a nitro, a hydroxyl,—CF3,—CN, or the like.

The term “aliphatic group” includes organic compounds characterized by straight or branched chains, typically having between 1 and 22 carbon atoms. Aliphatic groups include alkyl groups, alkenyl groups and alkynyl groups. In complex structures, the chains can be branched or cross-linked. Alkyl groups include saturated hydrocarbons having one or more carbon atoms, including straight-chain alkyl groups and branched-chain alkyl groups. Such hydrocarbon moieties can be substituted on one or more carbons with, for example, a halogen, a hydroxyl, a thiol, an amino, an alkoxy, an alkylcarboxy, an alkylthio, or a nitro group. Unless the number of carbons is otherwise specified, “lower aliphatic” as used herein means an aliphatic group, as defined above (e.g., lower alkyl, lower alkenyl, lower alkynyl), but having from one to six carbon atoms. Representative of such lower aliphatic groups, e.g., lower alkyl groups, are methyl, ethyl, n-propyl, isopropyl, 2-chloropropyl, n-butyl, sec-butyl, 2-aminobutyl, isobutyl, tert-butyl, 3-thiopentyl and the like. As used herein, the term “nitro” means—NO2; the term “halogen” designates—F, —Cl, —Br or—I; the term “thiol” means SH; and the term “hydroxyl” means—OH. Thus, the term “alkylamino” as used herein means an alkyl group, as defined above, having an amino group attached thereto. Suitable alkylamino groups include groups having 1 to about 12 carbon atoms, preferably from 1 to about 6 carbon atoms. The term “alkylthio” refers to an alkyl group, as defined above, having a sulfhydryl group attached thereto. Suitable alkylthio groups include groups having 1 to about 12 carbon atoms, preferably from 1 to about 6 carbon atoms. The term “alkylcarboxyl” as used herein means an alkyl group, as defined above, having a carboxyl group attached thereto. The term “alkoxy” as used herein means an alkyl group, as defined above, having an oxygen atom attached thereto. Representative alkoxy groups include groups having 1 to about 12 carbon atoms, preferably 1 to about 6 carbon atoms, e.g., methoxy, ethoxy, propoxy, tert-butoxy and the like. The terms “alkenyl” and “alkynyl” refer to unsaturated aliphatic groups analogous to alkyls, but which contain at least one double or triple bond respectively. Suitable alkenyl and alkynyl groups include groups having 2 to about 12 carbon atoms, preferably from 1 to about 6 carbon atoms.

The term “alkyl” includes saturated aliphatic groups, including straight-chain alkyl groups, branched-chain alkyl groups, cycloalkyl (alicyclic) groups, alkyl substituted cycloalkyl groups and cycloalkyl substituted alkyl groups. In certain embodiments, a straight chain or branched chain alkyl has 30 or fewer carbon atoms in its backbone, e.g., C1-C30 for straight chain or C3-C30 for branched chain. In certain embodiments, a straight chain or branched chain alkyl has 20 or fewer carbon atoms in its backbone, e.g., C1-C20 for straight chain or C3-C2O for branched chain, and more preferably 18 or fewer. Likewise, preferred cycloalkyls have from 4-10 carbon atoms in their ring structure and more preferably have 4-7 carbon atoms in the ring structure. The term “lower alkyl” refers to alkyl groups having from 1 to 6 carbons in the chain and to cycloalkyls having from 3 to 6 carbons in the ring structure.

Moreover, the term “alkyl” (including “lower alkyl”) as used throughout the specification and claims includes both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfate, sulfonato, sulfamoyl, sulfonamido, nitro, trifluoromethyl, cyano, azido, heterocyclyl, aralkyl, or an aromatic or hetero aromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl, e.g., having 1 to 3 separate or fused rings and from 6 to about 18 carbon ring atoms, e.g., phenylmefhyl(benzyl).

The term “amino,” as used herein, refers to an unsubstituted or substituted moiety of the formula—NRaRb, in which Ra and Rb are each independently hydrogen, alkyl, aryl, or heterocyclyl, or Ra and R, taken together with the nitrogen atom to which they are attached, form a cyclic moiety having from 3 to 8 atoms in the ring. Thus, the term “amino” includes cyclic amino moieties such as piperidinyl or pyrrolidinyl groups, unless otherwise stated. An “amino-substituted amino group” refers to an amino group in which at least one of Ra and Rb, is further substituted with an amino group.

The term “aromatic group” includes unsaturated cyclic hydrocarbons containing one or more rings. Aromatic groups include 5- and 6-membered single-ring groups which can include from zero to four heteroatoms, for example, benzene, pyrrole, furan, thiophene, imidazole, oxazole, thiazole, triazole, pyrazole, pyridine, pyrazine, pyridazine and pyrimidine and the like. The aromatic ring can be substituted at one or more ring positions with, for example, a halogen, a lower alkyl, a lower alkenyl, a lower alkoxy, a lower alkylthio, a lower alkylamino, a lower alkylcarboxyl, a nitro, a hydroxyl,—CF3, —CN, or the like.

The term “aryl” includes 5- and 6-membered single-ring aromatic groups that can include from zero to four heteroatoms, for example, unsubstituted or substituted benzene, pyrrole, furan, thiophene, imidazole, oxazole, thiazole, triazole, pyrazole, pyridine, pyrazine, pyridazine and pyrimidine and the like. Aryl groups also include polycyclic fused aromatic groups such as naphthyl, quinolyl, indolyl and the like. The aromatic ring can be substituted at one or more ring positions with such substituents, e.g., as described above for alkyl groups. Suitable aryl groups include unsubstituted and substituted phenyl groups. The term “aryloxy” as used herein means an aryl group, as defined above, having an oxygen atom attached thereto. The term “aralkoxy” as used herein means an aralkyl group, as defined above, having an oxygen atom attached thereto. Suitable aralkoxy groups have 1 to 3 separate or fused rings and from 6 to about 18 carbon ring atoms, e.g., O-benzyl.

The term “ceramic precursor” is intended include any compound that results in the formation of a ceramic material.

The term “chiral moiety” is intended to include any functionality that allows for chiral or stereoselective syntheses. Chiral moieties include, but are not limited to, substituent groups having at least one chiral center, natural and unnatural amino-acids, peptides and proteins, derivatized cellulose, macrocyclic antibiotics, cyclodextrins, crown ethers, and metal complexes.

The term “embedded polar functionality” is a functionality that provides an integral polar moiety such that the interaction with basic samples due to shielding of the unreacted silanol groups on the silica surface is reduced. Embedded polar functionalities include, but are not limited to carbonate, amide, urea, ether, thioefher, sulfinyl, sulfoxide, sulfonyl, thiourea, thiocarbonate, thiocarbamate, ethylene glycol, heterocyclic, triazole functionalities or carbamate functionalities such as disclosed in U.S. Pat. No. 5,374,755, and chiral moieties.

The language “chromatographically-enhancing pore geometry” includes the geometry of the pore configuration of the presently-disclosed materials, which has been found to enhance the chromatographic separation ability of the material, e.g., as distinguished from other chromatographic media in the art. For example, a geometry can be formed, selected or constructed, and various properties and/or factors can be used to determine whether the chromatographic separations ability of the material has been “enhanced,” e.g., as compared to a geometry known or conventionally used in the art. Examples of these factors include high separation efficiency, longer column life and high mass transfer properties (as evidenced by, e.g., reduced band spreading and good peak shape.) These properties can be measured or observed using art-recognized techniques. For example, the chromatographically-enhancing pore geometry of the present porous inorganic/organic hybrid materials is distinguished from the prior art materials by the absence of “ink bottle” or “shell shaped” pore geometry or morphology, both of which are undesirable because they, e.g., reduce mass transfer rates, leading to lower efficiencies.

Chromatographically-enhancing pore geometry is found in hybrid materials containing only a small population of micropores. A small population of micropores is achieved in hybrid materials when all pores of a diameter of about <34 A contribute less than about 110 m2/g to the specific surface area of the material. Hybrid materials with such a low micropore surface area (MSA) give chromatographic enhancements including high separation efficiency and good mass transfer properties (as evidenced by, e.g., reduced band spreading and good peak shape). Micropore surface area (MSA) is defined as the surface area in pores with diameters less than or equal to 34 A, determined by multipoint nitrogen sorption analysis from the adsorption leg of the isotherm using the BJH method. As used herein, the acronyms “MSA” and “MP A” are used interchangeably to denote “micropore surface area.”

The term “functionalizing group” includes organic functional groups which impart a certain chromatographic functionality to a chromatographic stationary phase.

The term “heterocyclic group” includes closed ring structures in which one or more of the atoms in the ring is an element other than carbon, for example, nitrogen, sulfur, or oxygen. Heterocyclic groups can be saturated or unsaturated and heterocyclic groups such as pyrrole and furan can have aromatic character. They include fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl,—CF3,—CN, or the like. Suitable heteroaromatic and heteroalicyclic groups generally will have 1 to 3 separate or fused rings with 3 to about 8 members per ring and one or more N, O or S atoms, e.g., coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl.

The term “metal oxide precursor” is intended include any compound that contains a metal and results in the formation of a metal oxide, e.g., alumina, silica, titanium oxide, zirconium oxide.

The term “monolith” is intended to include a collection of individual particles packed into a bed formation, in which the shape and morphology of the individual particles are maintained. The particles are advantageously packed using a material that binds the particles together. Any number of binding materials that are well known in the art can be used such as, for example, linear or cross-linked polymers of divinylbenzene, methacrylate, urethanes, alkenes, alkynes, amines, amides, isocyanates, or epoxy groups, as well as condensation reactions of organoalkoxysilanes, tetraalkoxysilanes, polyorganoalkoxysiloxanes, polyethoxysiloxanes, and ceramic precursors. In certain embodiments, the term “monolith” also includes hybrid monoliths made by other methods, such as hybrid monoliths detailed in U.S. Pat. No. 7,250,214; hybrid monoliths prepared from the condensation of one or more monomers that contain 0-99 mole percent silica (e.g., Si(¾); hybrid monoliths prepared from coalesced porous inorganic/organic particles; hybrid monoliths that have a chromatographically-enhancing pore geometry; hybrid monoliths that do not have a chromatographically-enhancing pore geometry; hybrid monoliths that have ordered pore structure; hybrid monoliths that have non-periodic pore structure; hybrid monoliths that have non-crystalline or amorphous molecular ordering; hybrid monoliths that have crystalline domains or regions; hybrid monoliths with a variety of different macropore and mesopore properties; and hybrid monoliths in a variety of different aspect ratios. In certain embodiments, the term “monolith” also includes inorganic monoliths, such as those described in G. Guiochon/7. Chromatogr. A 1168 (2007) 101-168.

The term “nanoparticle” is a microscopic particle/grain or microscopic member of a powder/nanopowder with at least one dimension less than about 100 nm, e.g., a diameter or particle thickness of less than about 100 nm (0.1 mm), which can be crystalline or noncrystalline. Nanoparticles have properties different from, and often superior to, those of conventional bulk materials including, for example, greater strength, hardness, ductility, sinterability, and greater reactivity among others. Considerable scientific study continues to be devoted to determining the properties of nanomaterials, small amounts of which have been synthesized (mainly as nano-size powders) by a number of processes including colloidal precipitation, mechanical grinding, and gas-phase nucleation and growth. Extensive reviews have documented recent developments in nano-phase materials, and are incorporated herein by reference thereto: Gleiter, H. (1989) “Nano-crystalline materials,” Prog. Mater. Sci. 33:223-315 and Siegel, R. W. (1993) “Synthesis and properties of nano-phase materials,” Mater. Sci. Eng. A168: 189-197. In certain embodiments, the nanoparticles comprise oxides or nitrides of the following: silicon carbide, aluminum, diamond, cerium, carbon black, carbon nanotubes, zirconium, barium, cerium, cobalt, copper, europium, gadolinium, iron, nickel, samarium, silicon, silver, titanium, zinc, boron, and mixtures thereof. In certain embodiments, the nanoparticles of the present invention are selected from diamonds, zirconium oxide (amorphous, monoclinic, tetragonal and cubic forms), titanium oxide (amorphous, anatase, brookite and rutile forms), aluminum (amorphous, alpha, and gamma forms), and boronitride (cubic form). In particular embodiments, the nanoparticles of the present invention are selected from nano-diamonds, silicon carbide, titanium dioxide (anatase form), cubic-boronitride, and any combination thereof. Moreover, in particular embodiments, the nanoparticles can be crystalline or amorphous. In particular embodiments, the nanoparticles are less than or equal to 100 mm in diameter, e.g., less than or equal to 50 mm in diameter, e.g., less than or equal to 20 mm in diameter.

Moreover, it should be understood that the nanoparticles that are characterized as dispersed within the composites of the invention are intended to describe exogenously added nanoparticles. This is in contrast to nanoparticles, or formations containing significant similarity with putative nanoparticles, that are capable of formation in situ, wherein, for example, macromolecular structures, such as particles, can comprise an aggregation of these endogenously created.

The term “substantially disordered” refers to a lack of pore ordering based on x-ray powder diffraction analysis. Specifically, “substantially disordered” is defined by the lack of a peak at a diffraction angle that corresponds to a d value (or d-spacing) of at least 1 nm in an x-ray diffraction pattern.

“Surface modifiers” include (typically) organic functional groups which impart a certain chromatographic functionality to a chromatographic stationary phase. The porous inorganic/organic hybrid materials possess both organic groups and silanol groups which can additionally be substituted or derivatized with a surface modifier.

The language “surface modified” is used herein to describe the composite material of the present invention that possess both organic groups and silanol groups which can additionally be substituted or derivatized with a surface modifier. “Surface modifiers” include (typically) organic functional groups which impart a certain chromatographic functionality to a chromatographic stationary phase. Surface modifiers such as disclosed herein are attached to the base material, e.g., via derivatization or coating and later crosslinking, imparting the chemical character of the surface modifier to the base material. In one embodiment, the organic groups of a hybrid material react to form an organic covalent bond with a surface modifier. The modifiers can form an organic covalent bond to the material's organic group via a number of mechanisms well known in organic and polymer chemistry including but not limited to nucleophilic, electrophilic, cycloaddition, free-radical, carbene, nitrene, and carbocation reactions. Organic covalent bonds are defined to involve the formation of a covalent bond between the common elements of organic chemistry including but not limited to hydrogen, boron, carbon, nitrogen, oxygen, silicon, phosphorus, sulfur, and the halogens. In addition, carbon-silicon and carbon-oxygen-silicon bonds are defined as organic covalent bonds, whereas silicon-oxygen-silicon bonds that are not defined as organic covalent bonds. A variety of synthetic transformations are well known in the literature, see, e.g., March, J. Advanced Organic Chemistry, 3rd Edition, Wiley, New York, 1985.

The term “porous material” is intended to include a member of a class of porous crosslinked polymers penetrated by pores through which solutions can diffuse. Pores are regions between densely packed polymer chains. In certain embodiments, pores include vacant space which is usually defined with a size of diameter. A pore on the chromatographic surface is usually open-ended so the molecule smaller than the size of the pore can reside in or pass through the pore and can be in any forms. Because pores in plurality provide large surface area as well as an alternative flow path, pores on the chromatographic surface can impact on retention time of analytes in chromatography and give chromatographic enhancements including high separation efficiency and good mass transfer properties (as evidenced by, e.g., reduced band spreading and good peak shape). Thus, the size distribution of pores usually is a key contributor for the benefit of the chromatography in the invention. Pores of chromatographic surface can be introduced with a synthesized polymer, for example, an ethylene bridged hybrid (BEH Technology™, Waters Corporation, Milford, Mass.,) with a silica that create an inert chemical structure.

The term “random ordering” is intended to include ordering in which individual units are joined randomly.

The term “hydrophilic interaction chromatography” or HILIC is intended to include a process employing a hydrophilic stationary phase and a hydrophobic organic mobile phase in which hydrophilic compounds are retained longer than hydrophobic compounds. In certain embodiments, the process utilizes a water-miscible solvent mobile phase. In certain embodiments, the term also includes ERLIC (electrostatic repulsion hydrophilic interaction chromatography), Cationic ERLIC and Anionic ERLIC.

The term “sorption” describes the ability of a material to take up and hold another material by absorption or adsorption.

The language “surface functionalized” is used herein to describe the composite material of the present invention that posses ion-exchange functional groups that impart a certain chromatographic functionality to the material.

The term as used herein, “sample” refers to a mixture of molecules that comprises at least an analyte molecule, e.g., glycoprotein, that is subjected to manipulation in accordance with the methods of the invention, including separating, analyzing, extracting, concentrating or profiling.

The term as used herein, “analysis” or “analyzing” are used interchangeably and refer to any of the various methods of separating, detecting, isolating, purifying, solubilizing, detecting and/or characterizing molecules of interest (e.g., glycoprotein). Examples include, but are not limited to, solid phase extraction, solid phase micro extraction, electrophoresis, mass spectrometry, e.g., HILIC, MALDI-MS or ESI, liquid chromatography, e.g., high performance, e.g., reverse phase, normal phase, or size exclusion, ion-pair liquid chromatography, liquid-liquid extraction, e.g., accelerated fluid extraction, supercritical fluid extraction, microwave-assisted extraction, membrane extraction, soxhlet extraction, precipitation, clarification, electrochemical detection, staining, elemental analysis, Edmund degradation, nuclear magnetic resonance, infrared analysis, flow injection analysis, capillary electrochromatography, ultraviolet detection, and combinations thereof.

The term as used herein, “profiling” refers to any of various methods of analysis which are used in combination to provide the content, composition, or characteristic ratio of biological molecules (e.g., glycoprotein) in a sample.

The term as used herein, “amide” is intended to include a derivative form of carboxylic acid in which the hydroxyl group has been replaced by an amine or ammonia. Due to the existence of strong electronegative atoms, oxygen and nitrogen, next to carbon, dipole moment is produced and the molecule with amide group presents polarity or hydrophilicity. In certain aspects of chromatography, amide group may be covalently bonded to the surface of the chromatographic core to impart a hydrophilicity to a chromatographic stationary phase.

The term as used herein, “ion pairing agent” is intended to include an ionic compound that imparts a certain hydrophobicity to other molecule, e.g. an analyte. Ion paring agent includes, but is not limited to, a hydrophobic counterion, for example, trifluoroacetic acid (TFA). Ion paring agent is capable of ion-pairing with the positively charged residues of analytes, such as positively charged amino acid residues of a peptide. Generally, ion pairing agent is added to the chromatography to reduce hydrophilicity or enhance hydrophobicity of the analyte molecules.

The term as used herein, “glycopeptide/glycoprotein” is a modified peptide/protein, during or after their synthesis, with covalently bonded carbohydrates or glycan.

The term as used herein, “glycan” is a compound comprising one or more of sugar units which commonly include glucose (Glc), galactose (Gal), mannose (Man), fucose (Fuc), N-acetylgalactosamine (GalNAc), N-acetylglucosamine (GlcNAc) and N-acetylneuraminic acid (NeuNAc) (Frank Kjeldsen, et al. Anal.Chem. 2003, 75, 2355-2361). The glycan moiety in glycoprotein is an important character to identify its function or cellular location. For example, most membrane bound proteins are glycoproteins for their intercellular or extracellular function. In other examples, a specific monoclonal antibody, e.g., trastuzumab (a commercial monoclonal antibody used for breast cancer treatment), is modified with specific glycan moiety.

### Chemicals and Reagents

Acetonitrile (Optima HPLC grade) was purchased from Fisher Scientific (Fair Lawn, N.J., USA). Ultrapure water was obtained from a Milli-Q Integral 5 water purification system from Millipore(Billerica, Mass., USA).

The following carbohydrates, L-(+)-arabinose, D-(+)-xylose, D-(−)-fructose, D-(+)-glucose, D-(+)-mannose, D-(+)-galactose, sucrose, D-(+)-maltose, lactose (fluka), L-(−)-glucose, L-(−)-sorbose, D-(+)-sorbose, D-(−)-ribose, D-psicose, D-(+)-trehalose, L-(−)-galactose, D-(−)-raffinose, D-altrose, D-(−)-tagatose, D-(−)-lyxose, D-(+)-turanose, D-allose, L-(+)-gulose, L-(+)-fructose, and palatinose, were purchased from Sigma-Aldrich (St. Louis, Mo., USA).

### Chromatographic Materials and Stationary Phases

In one aspect, the present invention provides a method for separation of carbohydrate tautomers from their mixtures using chromatographic materials and stationary phases described in WO2013173501.

In one aspect, the present invention uses a chromatographic stationary phase material for normal phase chromatography, high-pressure liquid chromatography, solvated gas chromatography, supercritical fluid chromatography, sub-critical fluid chromatography, carbon dioxide based chromatography, hydrophilic interaction liquid chromatography or hydrophobic interaction liquid chromatography represented by Formula 1: [X](W)a(Q)b(T)c(Formula 1). X can be a high purity chromatographic core composition having a surface including a silica core material, metal oxide core material, an inorganic-organic hybrid material or a group of block copolymers thereof. W can be absent and/or can include hydrogen and/or can include a hydroxyl on the surface of X. Q can be a functional group that minimizes retention variation over time (drift) under chromatographic conditions utilizing low water concentrations. T can include one or more hydrophilic, polar, ionizable, and/or charged functional groups that chromato graphically interact with the analyte. Additionally, b and c can be positive numbers, with the ratio 0.05<(b/c)<100, and a>0.

In another aspect, the present invention uses a chromatographic stationary phase material represented by Formula 1: [X](W)a(Q)b(T)c (Formula 1). X can be a chromatographic core material including a silica based, metal oxide based, or inorganic- organic hybrid based core surface. W can be absent and/or can include hydrogen and/or can include hydroxyl on the surface of X. Q can include one or more functional groups that essentially prevent chromatographic interaction between an analyte, and X and W, such that a first fraction of Q is bound to X and a section fraction of Q is polymerized. T can include one or more hydrophilic, polar, ionizable, and/or charged functional groups that chromatographically interact with the analyte, such that a third fraction of T is bound to Q, a fourth fraction of T is bound to X, and a fourth fraction of T is polymerized. Additionally, b and c can be positive numbers, with the ratio 0.05<(b/c)<100, and a>0.

In one or more embodiments, Q is represented by:

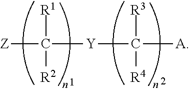

In one or more embodiments, n is an integer from 0-30 and n is an integer from 0-30. Each occurrence of R , R % RJ and R can independently represent hydrogen, fluoro, methyl, ethyl, n-butyl, t-butyl, i-propyl, lower alkyl, a protected or deprotected alcohol, a zwitterion, or a group Z. In some embodiments, group Z includes a surface attachment group having the formula (B1)x(R5)y(R6)zSi—, wherein xis an integer from 1-3, y is an integer from 0-2, z is an integer from 0-2, and x+y+z=3. Each occurrence of R5 and R6 can independently represent methyl, ethyl, ra-butyl, iso-butyl, teri-butyl, wo-propyl, thexyl, substituted or unsubstituted aryl, cyclic alkyl, branched alkyl, lower alkyl, a protected or deprotected alcohol, or a zwitterion group and B1 can represent a siloxane bond. In some embodiments, group Z includes an attachment to a surface organofunctional hybrid group through a direct carbon-carbon bond formation or through a heteroatom, ester, ether, thioether, amine, amide, imide, urea, carbonate, carbamate, heterocycle, triazole, or urethane linkage. In some embodiments, group Z includes an adsorbed, surface group that is not covalently attached to the surface of the material. Y can be an embedded polar functionality. In some embodiments, A represents a hydrophilic terminal group. In some embodiments, A represents hydrogen, fluoro, methyl, ethyl, n-butyl, t-butyl, i-propyl, lower alkyl, or group Z. In some embodiments, A represents a functionalizable group.

In one or more embodiments, T is represented by one of:

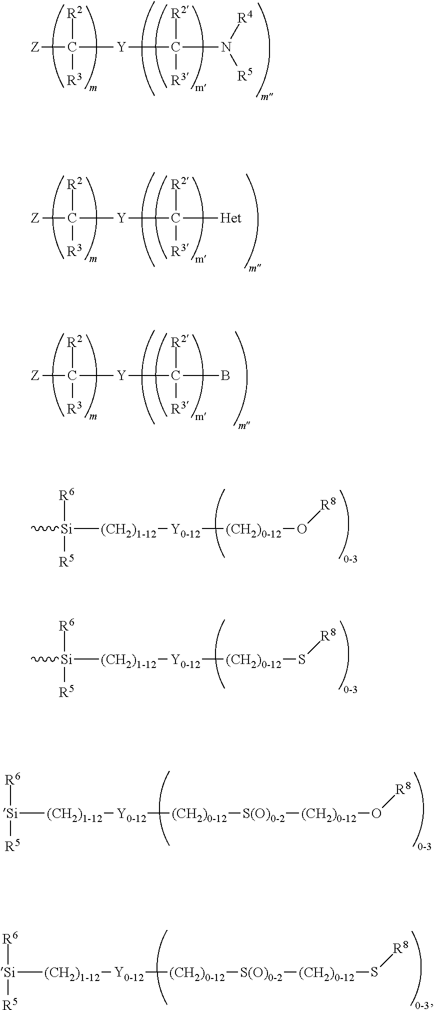

or a combination thereof. In some embodiments, m is an integer from 0-30; m′ is an integer from 0-30; and m″ is an integer from 0-3. In some embodiments, Z represents a surface attachment group having the formula (B¹)ₓ(R⁵)_{y}(R⁶)_{z}Si—, wherein x is an integer from 1-3, y is an integer from 0-2, z is an integer from 0-2, and x+y+z=3. Each occurrence of R⁵ and R⁶ can independently represent methyl, ethyl, re-butyl, wo-butyl, feri-butyl, iso-propyl, thexyl, substituted or unsubstituted aryl, cyclic alkyl, branched alkyl, lower alkyl, a protected or deprotected alcohol, or a zwitterion group. B¹ can represent a siloxane bond; where each of R⁷′ R⁷ and R⁷ represents hydrogen, methyl, ethyl, re-butyl, iso-butyl, iert-butyl, z′so-propyl, thexyl, phenyl, branched alkyl or lower alkyl. In some embodiments, Z represents an attachment to a surface organofunctional hybrid group through a direct carbon-carbon bond formation or through a heteroatom, ester, ether, thioether, amine, amide, imide, urea, carbonate, carbamate, heterocycle, triazole, or urethane linkage. In some embodiments, Z represents an adsorbed, surface group that is not covalently attached to the surface of the material.

In some embodiments, b and c are positive numbers, with a ratio 0.05<(b/c)<100, and a>0. In some embodiments, Q and T are different, whereas in other embodiments Q and T are the same. Q can include two or more different moieties, and T can include two or more different moieties. In some embodiments, the first, second, third, fourth, and fifth fraction are each independently about 0-100, 1-99, 5-95, 10-90, 20-80, 30-70, 40-60, 0, 5, 10, 15, 20, 25, 30, 35, 40, 45, 50, 55, 60, 65, 70, 75, 80, 85, 90, or 95%. In one or more embodiments, Q is non-polar. In some embodiments, Q comprises a borate or nitro functional group. In some embodiments, Q is represented by one of:

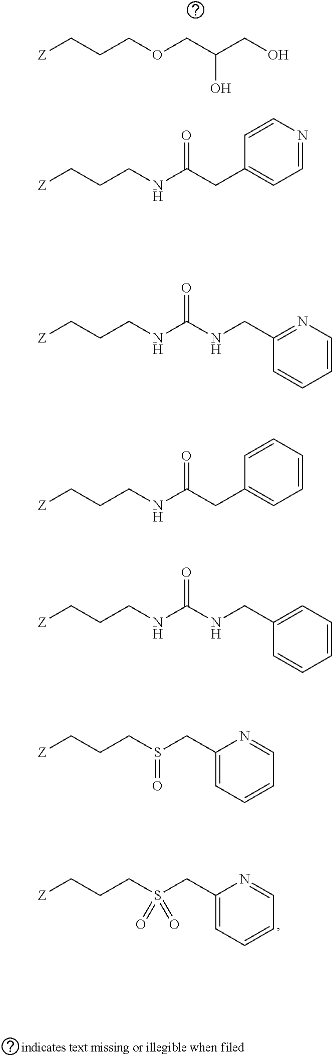

wherein Z can include a surface attachment group having the formula (B¹)ₓ(R⁵)_{y}(R⁶)_{z}Si—, wherein x is an integer from 1-3, y is an integer from 0-2, z is an integer from 0-2, and x+y+z=3. Each occurrence of R⁵ and R⁶ can independently represent methyl, ethyl, ra-butyl, /io-butyl, tert-butyl, iso-propyl, thexyl, substituted or unsubstituted aryl, cyclic alkyl, branched alkyl, lower alkyl, a protected or deprotected alcohol, or a zwitterion group, and B¹ can represent a siloxane bond.

In another embodiment, Z is an attachment to a surface organofunctional hybrid group through a direct carbon-carbon bond formation or through a heteroatom, ester, ether, thioether, amine, amide, imide, urea, carbonate, carbamate, heterocycle, triazole, or urethane linkage. In yet another embodiment, Z is an adsorbed, surface group that is not covalently attached to the surface of the material.

In some embodiments, T is represented by one of:

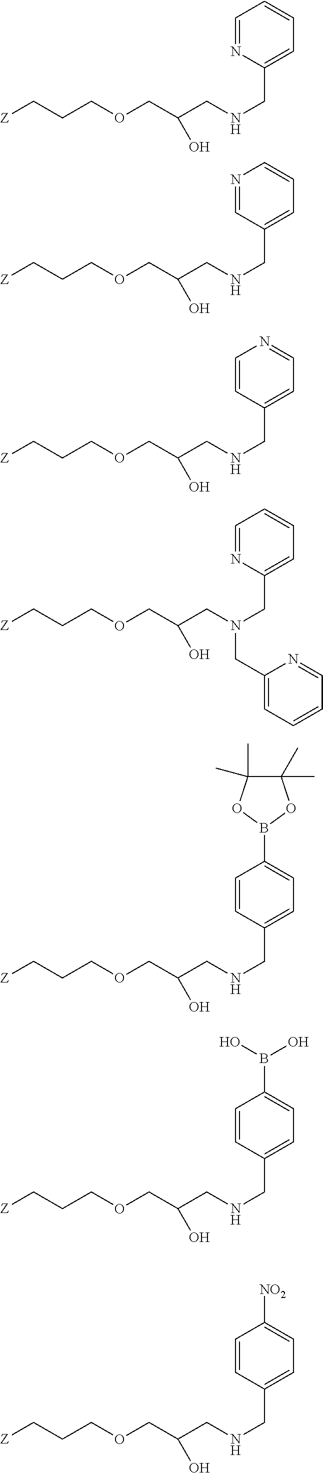

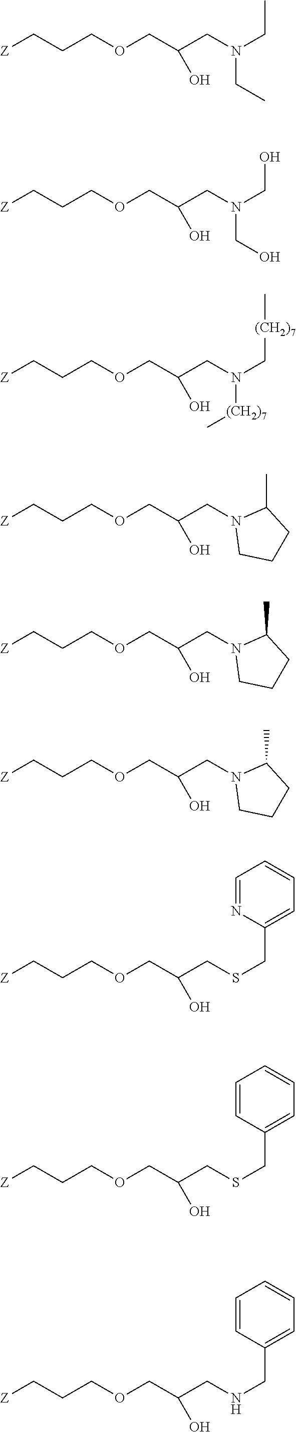

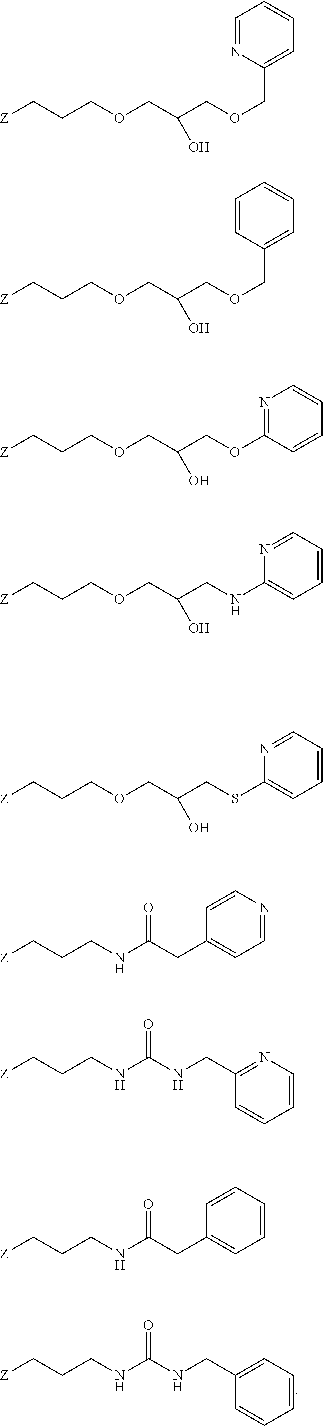

In some embodiments, Z includes a surface attachment group having the formula (B¹)ₓ(R⁵)_{y}(R⁶)_{z}Si—, wherein x is an integer from 1-3, y is an integer from 0-2, z is an integer from 0-2, and x+y+z=3. Each occurrence of R⁵ and R⁶ can independently represent methyl, ethyl, ra-butyl, iso-butyl, Zeri-butyl, wo-propyl, thexyl, substituted or unsubstituted aryl, cyclic alkyl, branched alkyl, lower alkyl, a protected or deprotected alcohol, or a zwitterion group, and B¹ can represent a siloxane bond. In some embodiments, Z is an attachment to a surface organofunctional hybrid group through a direct carbon-carbon bond formation or through a heteroatom, ester, ether, thioether, amine, amide, imide, urea, carbonate, carbamate, heterocycle, triazole, or urethane linkage. In some embodiments, Z is an adsorbed, surface group that is not covalently attached to the surface of the material.

In one or more embodiments of any of the above aspects, X is a high purity chromatographic material having a core surface that is subject to alkoxylation by a chromatographic mobile phase under chromatographic conditions. X can be a chromatographic material having a core surface that is subject to alkoxylation by a chromatographic mobile phase under chromatographic conditions. In some embodiments, the functional group including Q is a diol. The functional group including T can be an amine, an ether, a thioether, or a combination thereof. T can include a chiral functional group adapted for a chiral separation, Q can include a chiral functional group adapted for a chiral separation, or T and Q can both include a chiral functional group adapted for a chiral separation.

In one or more embodiments of the above aspects, the ratio b/c is about 0.05-75, 0.05-50, 0.1, 0.2, 0.3, 0.4, 0.5, 0.6, 0.7, 0.8, 0.9, 1, 5, 10, 15, 20, 25, 30, 35, 40, 45, 50, 60, 70, 80, or 90. In some embodiments, the surface of X does not include silica, and b=0 or c=0. In some embodiments, the combined surface coverage is greater than about 0.8, 0.9, 1.0, 1.1, 1.2, 1.3, 1.4, 1.5, 1.6, 1.7, 1.8, 1.9, 2.0, 2.1 , 2.2, 2.3, 2.4, 2.5, 2.6, 2.7, 2.8, 2.9, 3.0, 3.1, 3.2, 3.4, 3.5, 3.6, 3.7, 3.8, 3.9, 4, 5, 6, 7, or 8 μηιo\ddot{\acute{ι}}/ηι2.

In some embodiments, the core material consists essentially of a silica material. Optionally, the core material consists essentially of an organic-inorganic hybrid material or a superficially porous material. In one or more embodiments, the core material consists essentially of an inorganic material with a hybrid surface layer, a hybrid material with an inorganic surface layer, a surrounded hybrid layer, or a hybrid material with a different hybrid surface layer. The stationary phase material can optionally be in the form of a plurality of particles, a monolith, or a superficially porous material. In some embodiments the stationary phase material does not have chromatographically enhancing pore geometry whereas in other embodiments the stationary phase material has chromatographically enhancing pore geometry. The stationary phase material can be in the form of a spherical material, non-spherical material (e.g., including toroids, polyhedron). In certain embodiments, the stationary phase material has a highly spherical core morphology, a rod shaped core morphology, a bent-rod shaped core morphology, a toroid shaped core morphology; or a dumbbell shaped core morphology. In certain embodiments, the stationary phase material has a mixture of highly spherical, rod shaped, bent rod shaped, toroid shaped, or dumbbell shaped morphologies.

In some embodiments, the stationary phase material has a surface area of about 25 to 1100 m2/g, about 150 to 750 m2/g, or about 300 to 500 m2/g. In some embodiments, the stationary phase material has a pore volume of about 0.2 to 2.0 cm3/g, or about 0.7 to 1.5 cm/g. In some embodiments, the stationary phase material has a micropore surface area of less than about 105 m 2/g, less than about 80 m 2/g, or less than about 50 m2/g. The stationary phase material can have an average pore diameter of about 20 to 1500 A, about 50 to 1000 A, about 60 to 750 A, or about 65 to 200 A. In some embodiments, the plurality of particles have sizes between about 0.2 and 100 microns, between about 0.5 and 10 microns, or between about 1.5 and 5 microns.

In one or more embodiments, X includes a silica core, c=0, and Q has a combined surface coverage of >2.5, 2.6, 2.7, 2.8, 2.9, 3.0, 3.5, 4.0, 4.5, or 5 μηιo\ddot{\acute{ι}}/ηι2; or X includes a non-silica core or a silica-organic hybrid core, c=0, and Q has a combined surface coverage of >0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or 5 μηιo\ddot{\acute{ι}}/ηι2; or b>0, c>0, and Q has a combined surface coverage of >0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or 5 μηιo\ddot{\acute{ι}}/πι2.

In some embodiments, the chromatographic stationary phase includes radially adjusted pores, non-radially adjusted pores, ordered pores, non-ordered pores, monodispersed pores, non-monodispersed pores, smooth surfaces, rough surfaces or combinations thereof. In one or more embodiments, T has one ionizable group, T has more than one ionizable group, T has two or more ionizable groups of the same p a, or T has two or more ionizable group of different pKa.

In yet another aspect, the present disclosure provides a method for preparing a stationary phase according to any of the materials of the present disclosure. The method includes oligomerizing a silane coupling agent having a pendant reactive group. The method further includes reacting a core surface with the oligomerized silane coupling agent. The method further includes reacting a second chemical agent including one or more hydrophilic, polar, ionizable, and/or charged functional groups with the pendant reactive group. The method further includes neutralizing any remaining unreacted pendant reactive groups, thereby producing a stationary phase according to the present disclosure.

In one or more embodiments, Q is derived from a reagent having one or the following structures:

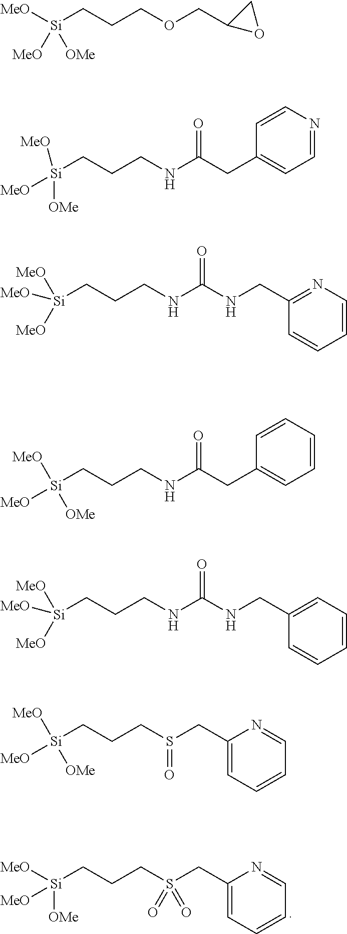

In some embodiments, T is derived from a reagent having one or the following structures:

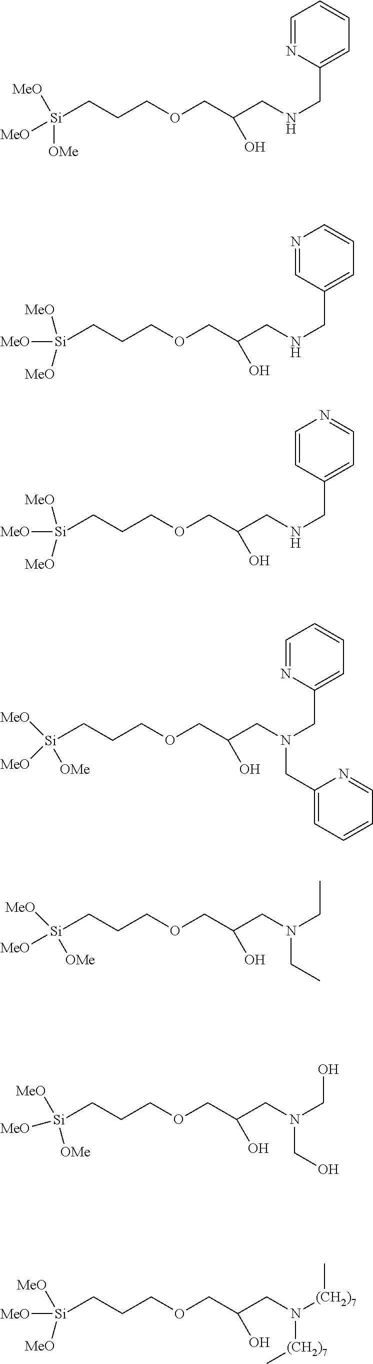

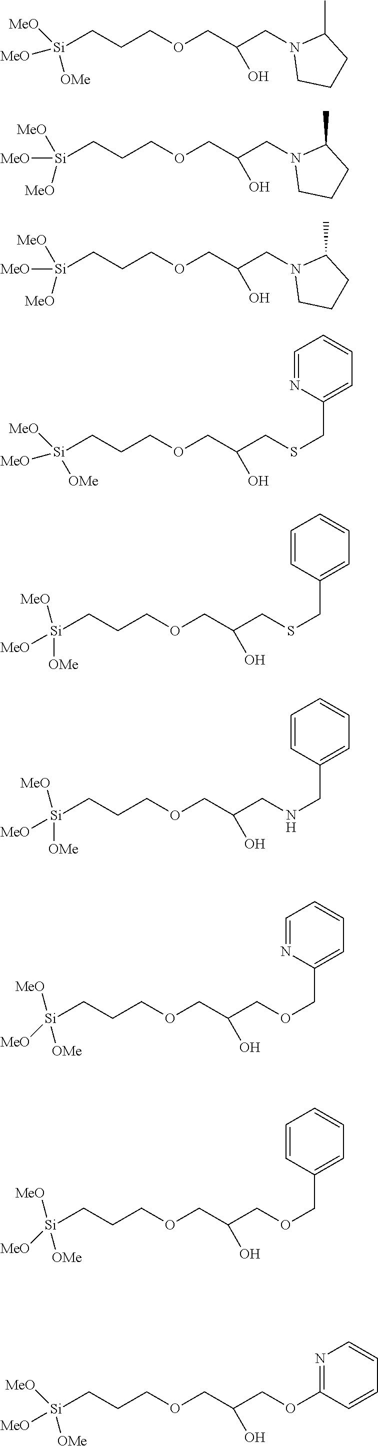

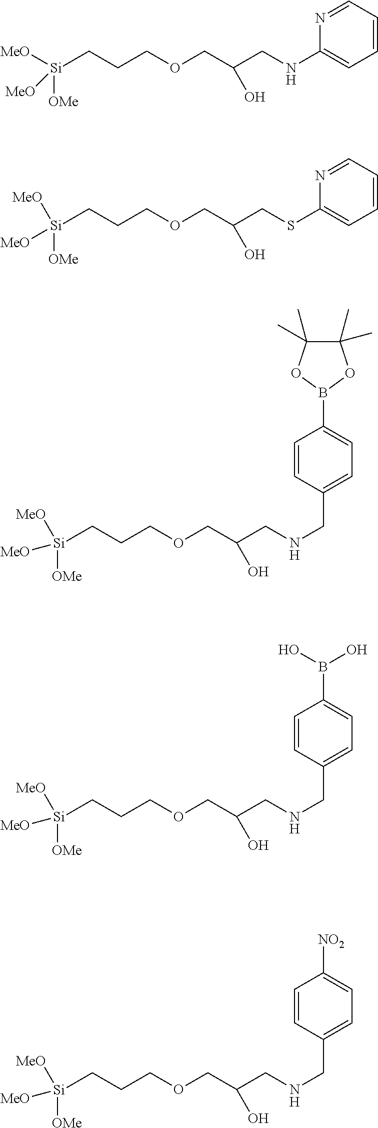 [SMILES: CO[Si](CCCOCC(O)CNc1ccccn1)(OC)OC ; CO[Si](CCCOCC(O)CSc1ccccn1)(OC)OC ; CO[Si](CCCOCC(O)CNCc1ccc(B2OC(C)(C)C(C)(C)O2)cc1)(OC)OC ; CO[Si](CCCOCC(O)CNCc1ccc(B(O)O)cc1)(OC)OC ; CO[Si](CCCOCC(O)CNCc1ccc([N+](=O)[O-])cc1)(OC)OC]

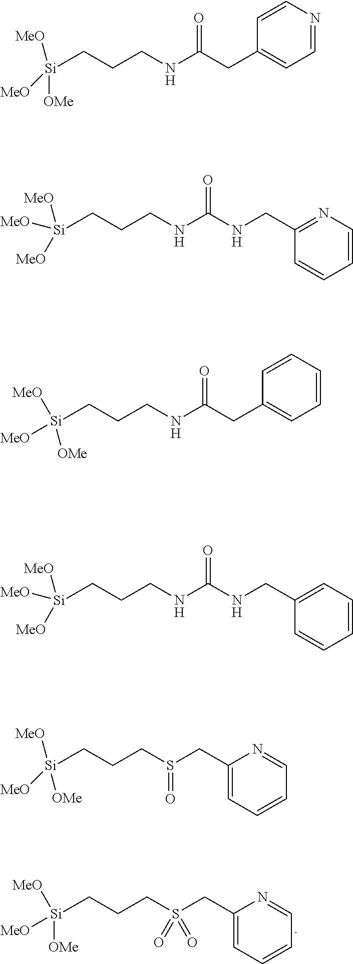 [SMILES: CO[Si](CCCNC(=O)Cc1ccncc1)(OC)OC ; CO[Si](CCCNC(=O)NCc1ccccn1)(OC)OC ; CO[Si](CCCNC(=O)Cc1ccccc1)(OC)OC ; CO[Si](CCCNC(=O)NCc1ccccc1)(OC)OC ; CO[Si](CCCS(=O)Cc1ccccn1)(OC)OC ; CO[Si](CCCS(=O)(=O)Cc1ccccn1)(OC)OC]

In some embodiments, T includes one of the following structures:

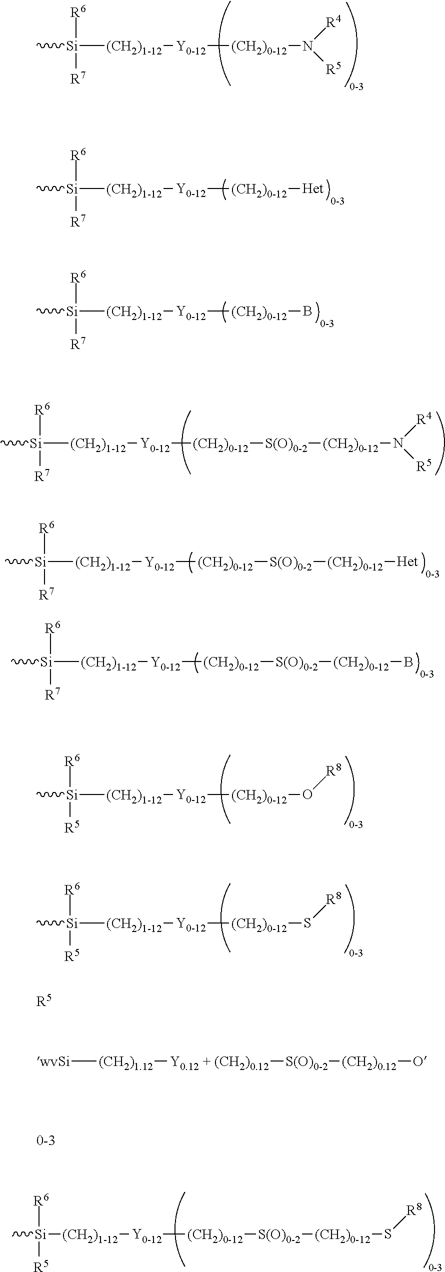

In some embodiments, Y includes one of the following structures:

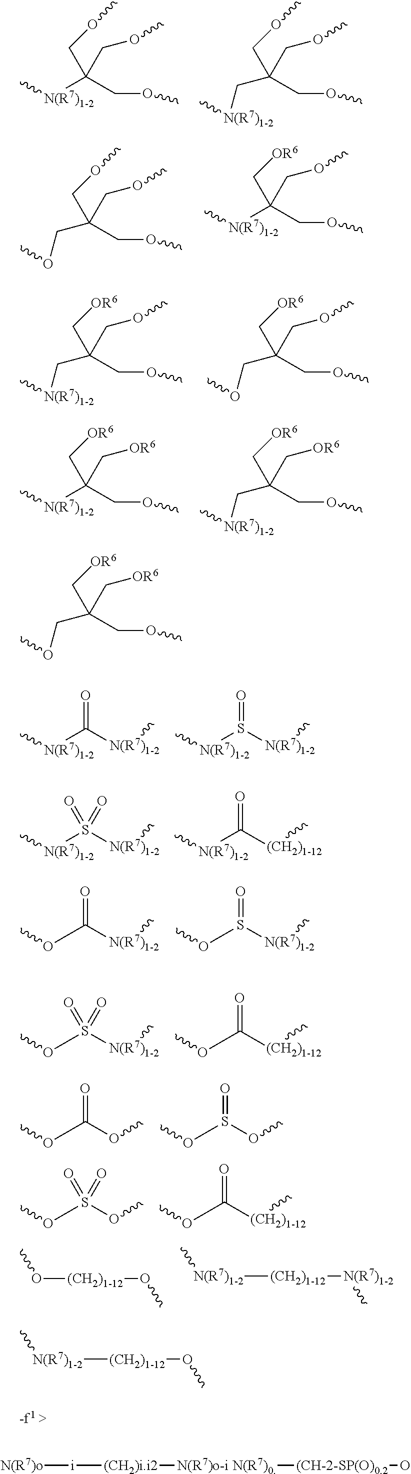

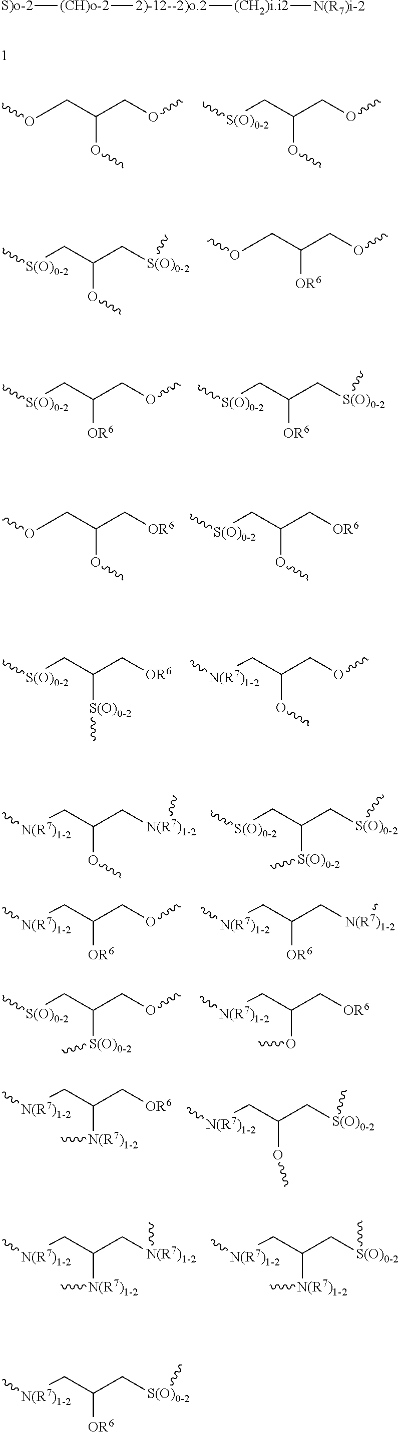

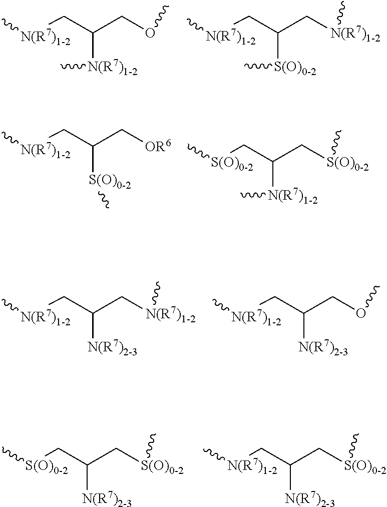

T can be derived from a reagent represented by:

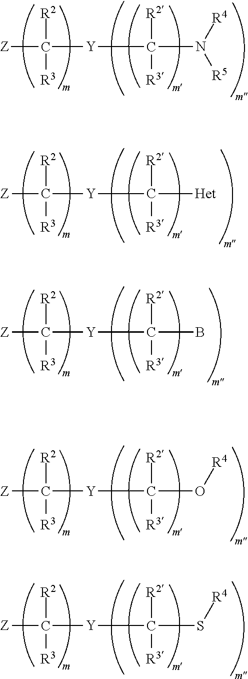

combination thereof, wherein m is an integer from 0-30; m′ is an integer from 0-30; and m″ is an integer from 0-3. Z can represent a chemically reactive group including:

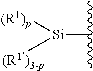

—OH, —OR, amine, alkylamine, dialkylamine, isocyanate, acyl chloride, triflate, isocyanate, thiocyanate, imidazole carbonate, NHS-ester, carboxylic acid, ester, epoxide, alkyne, alkene, azide, —Br, —CI, or —I. Y can be an embedded polar functionality. Each occurrence of R¹ can independently represent a chemically reactive group on silicon, including (but not limited to) —H, —OH, —OR⁶, dialkylamine, triflate, Br, CI, I, vinyl, alkene, or —(CH₂)ₘ Q; Each occurrence of Q can be —OH, —OR⁶, amine, alkylamine, dialkylamine, isocyanate, acyl chloride, triflate, isocyanate, thiocyanate, imidazole carbonate, NHS-ester, carboxylic acid, ester, epoxide, alkyne, alkene, azide, —Br, —CI, or —I. p can be an integer from 1-3. Each occurrence of R¹ can independently represent F, Ci-Ci8 alkyl, C₂-C18 alkenyl, C₂-C18 alkynyl, C3-C18 cycloalkyl, Q-C is heterocycloalkyl, C5-Ci8 aryl, C5-C18 aryloxy, or C₁-C18 heteroaryl, fluoroalkyl, or fluoroaryl. Each occurrence of R² and R³ can independently represent hydrogen, C₁-C18 alkyl, C₂-C18 alkenyl, C₂-C18 alkynyl, C3-C18 cycloalkyl, C₁-C18 heterocycloalkyl, C5-C18 aryl, C5-C18 aryloxy, or Ci-Ci8 heteroaryl, —Z, or a group having the formula —Si(R′)bR″_{a 0r}—C(R′)bR″a- The variables a and b can each represent an integer from 0 to 3, provided that a+b=3. R′ can represent a C1-C6 straight, cyclic or branched alkyl group. R″ can be a functionalizing group selected from the group consisting of alkyl, alkenyl, alkynyl, aryl, cyano, amino, diol, nitro, ester, a cation or anion exchange group, an alkyl or aryl group containing an embedded polar functionality and a chiral moiety. R⁴ can represent hydrogen, C1-C18 alkyl, C₂-C18 alkenyl, C₂-C18 alkynyl, C3-C18 cycloalkyl, C1-C18 heterocycloalkyl, C5-C18 aryl, substituted aryl, C5-C18 aryloxy, or Ci-Cie heteroaryl. R⁵ can represent hydrogen, C₁-C18 alkyl, C₂-C18 alkenyl, C₂-C18 alkynyl, C3-C18 cycloalkyl, Ci-Cis heterocycloalkyl, C5-C18 aryl, substituted aryl, C5-C18 aryloxy, or C1-C18 heteroaryl. Each occurrence of R⁶ can independently represent C1-C18 alkyl, C₂-C18 alkenyl, C₂-C18 alkynyl, C3-C18 cycloalkyl, Ci-Cie heterocycloalkyl, C5-C18 aryl, C5-C18 aryloxy, or Ci-Cie heteroaryl. Het can represent a monocyclic or bicyclic heterocyclic or heteroaryl ring system including at least one nitrogen atom. B can represent an acidic ionizable modifier.

Reference is made to WO2013173501 for complete description of chromatographic materials and stationary phases described above.

In another aspect, the present invention provides a method for separation of carbohydrate tautomers from their mixtures using poly-amide bonded HILIC stationary phases materials.

In one aspect, a porous material comprising a copolymer comprising at least one hydrophilic monomer and a poly-amide bonded phase, wherein the average pore diameter is greater than or equal to about 200 Å, greater than or equal to about 250 Å, greater than or equal to about 300 Å, or greater than or equal to about 450 Å.

In certain aspect, the porous material comprises a porous particle that comprises said copolymer. In another certain aspect, the porous material comprises a porous monolith that comprises said copolymer.

In another aspect, The hydrophilic monomer is 3-methacryloxypropyltrichlorosilane, 3-methacryloxypropylmethyldichlorosilane, 3-methacryloxypropyldimethylchlorosilane, 3-methacryloxypropyltrimethoxysilane, 3-methacryloxypropylmethyldimethoxysilane, 3-methacryloxypropyldimethylmethoxysilane, 3-methacryloxypropyltriethoxysilane, 3-methacryloxypropylmethyldiethoxysilane, 3-methacryloxypropyldimethylethoxysilane, 3-acryloxypropyltrichlorosilane, 3-acryloxypropylmethyldichlorosilane, 3-acryloxypropyldimethylchlorosilane 3-acryloxypropyltrimethoxysilane, 3-acryloxypropylmethyldimethoxysilane, 3-acryloxypropyldimethylmethoxysilane, 3-acryloxypropyltriethoxysilane, 3-acryloxypropylmethyldiethoxysilane, 3-acryloxypropyldimethylethoxysilane, styrylethyltrichlorosilane, styrylethylmethyldichlorosilane, styrylethyldimethylchlorosilane, styrylethyltrimethoxysilane, styrylethylmethyldimethoxysilane, styrylethyldimethylmethoxysilane, styrylethyltriethoxysilane, styrylethylmethyldiethoxysilane, styrylethyldimethylethoxysilane, vinyltriethoxysilane, vinyltrimethoxysilane, N-(3-acryloxy-2-hydroxypropyl)-3-aminopropyl triethoxysilane, (3-acryloxypropyl) trimethoxysilane, O-(methacryloxyethyl)-N-(triethoxysilylpropyl)urethane, N-(3-methacryloxy-2-hydroxypropyl)-3-aminopropyl triethoxysilane, methacryloxy methyltriethoxysilane, methacryloxymethyl trimethoxysilane, methacryloxypropy methyldiethoxysilane, methacryloxypropyl methyldimethoxysilane, methacryl oxypropyltris (methoxyethoxy)silane, 3-(N-styrylmethyl-2-aminoethylamino) propyltrimethoxysilane hydrochloride,

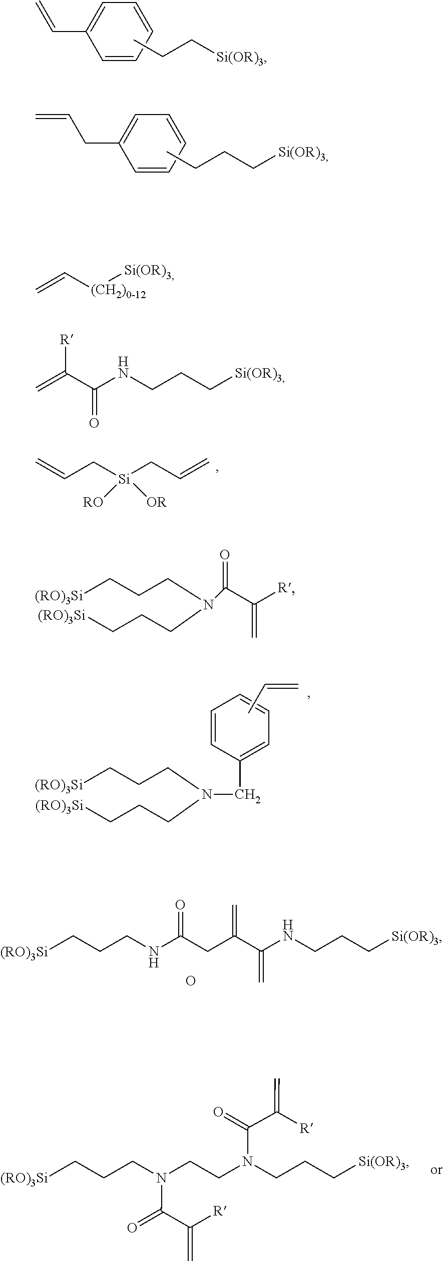

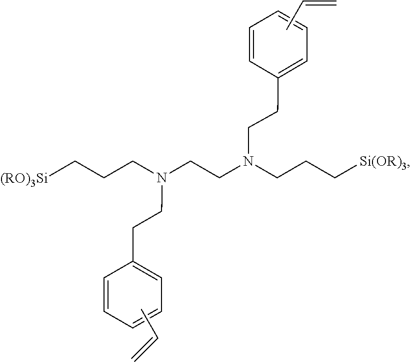

In yet another aspect, the poly-amide bonded phase is derived from acrylamide, divinylbenzene, styrene, ethylene glycol dimethacrylate, 1-vinyl-2-pyrrolidinone and tert-butylmethacrylate, acrylamide, methacrylamide, N,N′-(1,2-dihydroxyethylene)bisacrylamide, N,N′-ethylenebisacrylamide,N,N′-methylenebisacrylamide, butyl acrylate, ethyl acrylate, methyl acrylate, 2-(acryloxy)-2-hydroxypropyl methacrylate , N,N-bis(2-cyanoethyl)acrylamide, N-acryloyltris(hydroxymethyl)aminomethane, 3-(acryloxy)-2-hydroxypropyl methacrylate, trimethylolpropane triacrylate, trimethylolpropane ethoxylate triacrylate, tris[(2-acryloyloxy)ethyl] isocyanurate, acrylonitrile, methacrylonitrile, itaconic acid, methacrylic acid, trimethylsilylmethacrylate, N-[tris(hydroxymethyl)methyl]acrylamide, (3-acrylamidopropyl)trimethylammonium chloride, [3-(methacryloylamino)propyl]dimethyl(3-sulfopropyl)ammonium hydroxide inner salt,

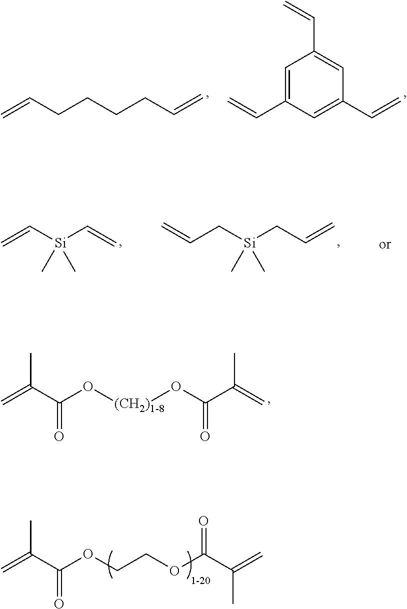

In certain aspect of the invention, the porous material comprises a second poly-amide bonded phase. In particular aspect, the second poly-amide bonded phase is derived from N,N-methylenebisacrylamide, N,N-ethylenebisacrylamide, N,N-propylenebisacrylamide, N,N-butylenebisacrylamide, N,N′-(1,2-dihydroxyethylene)bisacrylamide, or 1,4-bis(acryloyl)piperazine.

In other aspect of the invention, the first poly-amide bonded phase is present in about 35 to about 99 mole % of the total poly-amide bonded phases and the second poly-amide bonded phase is present in about 65 to about 1 mole % of the total poly-amide bonded phases.

In certain aspect, the porous material has a median pore diameter of about 100 Å to about 1000 Å, of about 300 Å to about 800 Å, or of about 300 Å to about 550 Å. In particular aspect, the porous material has a median pore diameter of about 300 Å.

In another certain aspect, the nitrogen content of the porous material is from about 0.5% N to about 20% N, from about 1% N to about 10% N, from about 2% N to about 10% N, or from about 4% N to about 10% N.

According to one embodiment, the present invention provides a method for removing or isolating a component from a mixture comprising: contacting the mixture with a chromatographic material comprising the porous material, to thereby remove or isolate the component from the mixture.

In certain embodiments according the method of the invention, the porous material is a poly(divinylbenzene-co-N-vinylcaprolactam) copolymer. In another certain aspect according to the method of the invention, the component is a biological material. In yet another certain aspect, the biological material is an intact protein, a denatured protein, a modified protein, an oligonucleotide, a modified oligonucleotide, a single-stranded oligonucleotide, a double-stranded oligonucleotide, DNA, RNA, or a peptide. In particular aspect, the biological material is an inclusion body, a biological fluid, a biological tissue, a biological matrix, an embedded tissue sample, or a cell culture supernatant.

In another certain aspect, the stationary phase is fully porous or superficially porous. In related aspect, the average diameter of said pores is greater than or equal to about 200 Å, greater than or equal to about 250 Å, greater than or equal to about 300 Å, or greater than or equal to about 450 Å. In another related aspect, the average diameter of said pores is from about 1 to about 50 Å, from about 5 to about 40 Å, or from about 10 to about 30 Å.

In another aspect of the invention, the stationary phase material comprises an organic-inorganic hybrid core comprising an aliphatic bridged silane. In related aspect, an aliphatic group of the aliphatic bridged silane is ethylene.

In certain aspect, the stationary phase material is in one or more forms of particles. In particular aspect, the average diameter of the particles is from about 0.1 μm and about 500 μm, from about 1 μm and about 100 μm, or from about 1 μm and about 10 μm. In another certain aspect, the stationary phase material further comprises a porous monolith.

In another certain aspect, the stationary phase material is fully porous or superficially porous. In related aspect, the average diameter of said pores is greater than or equal to about 200 Å, greater than or equal to about 250 Å, greater than or equal to about 300 Å, or greater than or equal to about 450. In another related aspect, the average diameter of said pores is from about 1 to about 50 Å, from about 5 to about 40 Å, or from about 10 to about 30 Å.

In another aspect of the invention, the stationary phase material comprises an organic-inorganic hybrid core comprising an aliphatic bridged silane. In related aspect, an aliphatic group of the aliphatic bridged silane is ethylene.

In certain aspect, the stationary phase material is in one or more forms of particles. In particular aspect, the average diameter of the particles is from about 0.1 μm and about 500 μm, from about 1 μm and about 100 μm, or from about 1 μm and about 10 μm. In another certain aspect, the stationary phase material further comprises a porous monolith.

Reference is made to WO2013173501 for complete description of chromatographic materials and stationary phases described above.

### Standard Sample Preparation

In certain embodiments, the method of the invention uses 1 mg/mL stock solutions of each of the studied carbohydrates in acetonitrile-water (75:25, v/v).

### Instrumentation and Chromatographic Conditions

In certain embodiments, the method of the invention were performed on an existing chromatography platforms such as commercially available chromatography systems including ACQUITY Ultra Performance Liquid Chromatographic (UPLC) system from Waters Corporation (Milford, Mass., USA). The system was comprised of a quaternary solvent manager (QSM), a flow through needle (FTN) sample manage, a column manager (CM-A) and either an evaporative light scattering (ELS) detector or a refractive index (RI) detector.

In certain embodiments, the UPLC system was equipped with one of the following columns: ACQUITY UPC²™ Torus DEA (diethylamine) column (50 mm×2.1 mm i.d., particle size 1.7 μm), ACQUITY UPC²™ BEH 2-EP (2-ethylpyridine) column (50 mm×2.1 mm i.d., particle size 1.7 μm), ACQUITY UPC²™ Torus Diol column (50 mm×2.1 mm i.d., particle size 1.7 μm), ACQUITY UPC²™ Torus 2-PIC (2-picolylamine) column (50 mm×2.1 mm i.d., particle size 1.7 μm), ACQUITY UPLC Glycan BEH Amide column (50 mm×2.1 mm i.d., particle size 1.7 μm) all from Waters Corporation, FLARE HILIC column (2.1 mm×100 mm i.d., particle size, 3.6 μm) from Diamond Analytics, Orem, Utah, an apHera™ NH2 Polymer column (4.6 mm×150 mm i.d., particle size, 5.0 μm) from Supelco Analytical, Bellefonte, Pa. or a Sugar SZ5533 column (150 mm×4.6 mm, i.d., particle size 6.0 μm) from Showa Denko K.K., Tokyo, Japan.

In certain embodiments, the invention provides a method of chromatographically separating carbohydrate tautomeric species using a chromatographic device with a mobile phase of acetonitrile and water at various concentrations (v/v), preferably, a mixture of 92% acetonitrile and 8% water using an ACQUITY UPC2™ Torus Diol column.

In certain embodiments, the invention provides a method of chromatographically separating carbohydrate tautomeric species using a chromatographic device with a mobile phase of acetone and water at various concentrations (v/v).

In certain embodiments, the method of the invention were performed using a chromatographic device with an injection volume ranging between 0.5 and 2 μL depending upon the column and the mobile phase flow rate used ranged between 0.2 and 0.6 mL/min depending upon the column.

In certain embodiments, the method of the invention were performed using a chromatographic device with an ACQUITY UPC2™ Torus Diol column at various temperatures between 10 and 60° C., preferably, at a temperature of 20° C.

In certain embodiments, the method of the invention were performed using an evaporative light scattering (ELS) detector with a nitrogen gas flow rate between 20 and 50 psi and a drift tube temperature between 40 and 60° C., preferably, a nitrogen gas flow rate of 30 psi and a drift tube temperature of 55° C.

In yet another embodiments, the method of the invention uses an existing chromatography platforms such as commercially available chromatography systems with a column packed with a porous inorganic/organic hybrid material, comprising porous inorganic/organic hybrid particles having a chromatographically-enhancing pore geometry, wherein the organic portion is selected from the group consisting of siloxanes and silanes, wherein the hybrid material has the formula SiO2/(R2 pR4qSiOt)n or SiO2/[R6(R2 rSiOt)m]n wherein R2 and R4 are independently C1-C18 aliphatic or aromatic moieties, R6 is a substituted or unsubstituted C1-C18 alkylene, alkenylene, alkynylene or arylene moiety bridging two or more silicon atoms, p and q are 0, 1 or 2, provided that p+q=1 or 2, and that when p+q=1, t=1.5, and when p+q=2, t=1; r is 0 or 1, provided that when r=0, t=1.5, and when r=1, t=1; m is an integer greater than or equal to 2, and n is a number from 0.03 to 1.

### SOFTWARE

In certain embodiments, Empower 3.0 Chromatography Data Software (Waters Corporation) was used to control the ACQUITY UPLC system, and to record and interpret the chromatograms.

### Separation of Carbohydrate Tautomers

In one embodiment, the invention provides a method of chromatographically separating tautomeric species of mono- and disaccharides tautomeric species and their mixtures.

In another embodiment, the invention provides a method of chromatographically separating tautomeric species of arabinose, xylose, fructose, mannose, galactose, sucrose, glucose, lactose, maltose, sorbose, ribose, psicose, trehalose, raffinose, altrose, tagatose, lyxose, turanose, allose, gulose, palatinose, or mixtures thereof.

In certain embodiments, the invention provides a method of chromatographically monitoring mutarotation of D-(+)-glucose tautomers in water.

In another embodiment, the invention provides a method of chromatographically separating tautomeric species of arabinose.

In another embodiment, the invention provides a method of chromatographically separating tautomeric species of xylose.

In another embodiment, the invention provides a method of chromatographically separating tautomeric species of fructose.

In another embodiment, the invention provides a method of chromatographically separating tautomeric species of fructose tautomers including β-D-fructopyranose, β-D-fructofuranose, α-D-fructopyranose, and α-D-fructofuranose.

In another embodiment, the invention provides a method of chromatographically separating tautomeric species of mannose.

In another embodiment, the invention provides a method of chromatographically separating tautomeric species of galactose.

In another embodiment, the invention provides a method of chromatographically separating tautomeric species of glucose.

In another embodiment, the invention provides a method of chromatographically separating tautomeric species of glucose tautomers including β-D-glucopyranose, and α-D-glucopyranose.

In another embodiment, the invention provides a method of chromatographically separating tautomeric species of lactose.

In another embodiment, the invention provides a method of chromatographically separating tautomeric species of maltose.

### EXAMPLES

The present invention may be further illustrated by the following non-limiting examples describing the methods of the invention.

Example 1

The following carbohydrate were dissolved in acetonitrile-water (75:25, v/v) to form a stock solution of 1 mg/mL: L-(+)-arabinose, D-(+)-xylose, D-(−)-fructose, D-(+)-glucose, D-(+)-mannose, D-(+)-galactose, sucrose (disaccharide), D-(+)-maltose (disaccharide), and Lactose (disaccharide).

Each of these stock solutions was injected in an UPLC system with an ACQUITY UPC2™ Torus DEA (diethylamine) column at a column temperature of 20° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid.

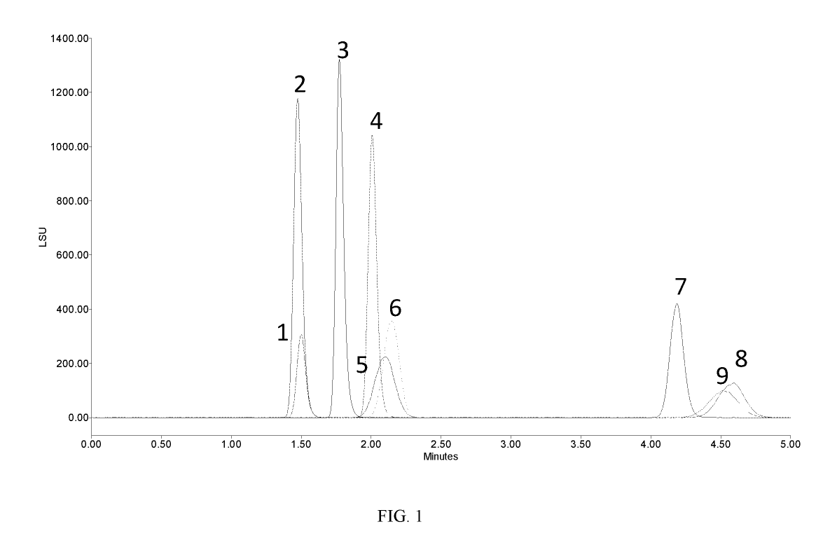

FIG. 1 shows overlaid chromatograms where each carbohydrate is a single peak. Even though some of the peaks are broad, no resolution of the individual tautomers are present.

### Example 2

Each of the stock solutions from Example 1 was injected in an UPLC system with an ACQUITY UPC2™ BEH 2-EP (2-ethylpyridine) column at a column temperature of 20° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid.

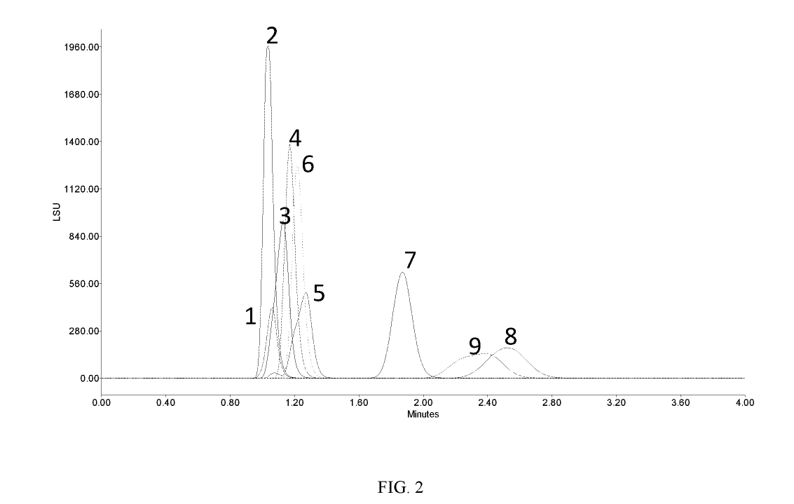

FIG. 2 shows overlaid chromatograms where each carbohydrate is a single peak. Even though some of the peaks are broad, in general no resolution of the individual tautomers are present. The exception to this is that a small peak is appearing in front of Peak 5 (galactose).

### Example 3

Each of these stock solutions from Example 1 was injected in an UPLC system with an ACQUITY UPC2™ Torus 2-PIC (2-picolylamine) column at a column temperature of 50° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid.

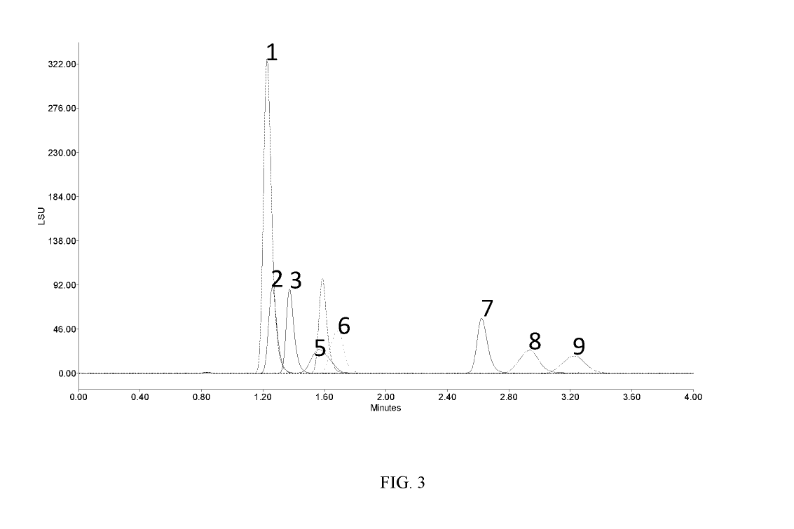

FIG. 3 shows overlaid chromatograms where each carbohydrate is a single peak. Note that the temperature is significantly higher in this example. This prevents the resolution of the tautomers. If the temperature is dropped by 10° C. (Figure Four), to 40, the peaks start to show significant peak broadening (peaks 8 and 9) and peak splitting (peaks 5 and 6) due to the tautomeric resolution.

### Example 4

Each of the stock solutions from Example 1 was injected in an UPLC system with an ACQUITY UPC2™ Torus 2-PIC (2-picolylamine) column at a column temperature of 40° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid.

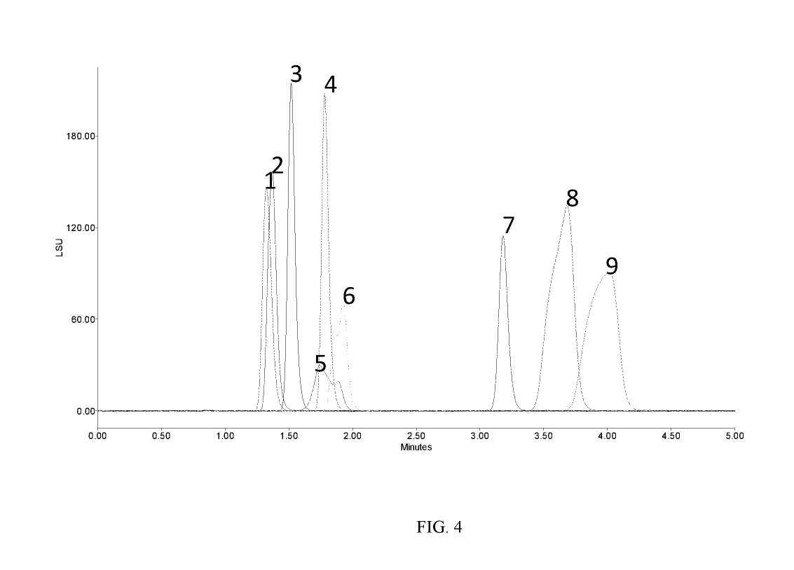

FIG. 4 shows broadened peaks and some peak splitting due to the resolution of the tautomers, the overall separation of the tautomers is poor.

### Example 5

Each of the stock solutions from Example 1 was injected in an UPLC system with an apHera™ NH2 Polymer column (a column commercialized for the analysis of mono- and di-saccharides) at a column temperature of 20 and 15° C., eluent consisting of acetonitrile and water (65/35) at a flow rate of 0.2 mL/min with ELS detection at a nitrogen pressure of 30 psi and a drift tube temperature of 55° C. Individual injections of each carbohydrate are overlaid. This demonstrates that a column that gives only a single peak for the combined tautomers cannot be made to separate the tautomers by decreasing the column temperature.

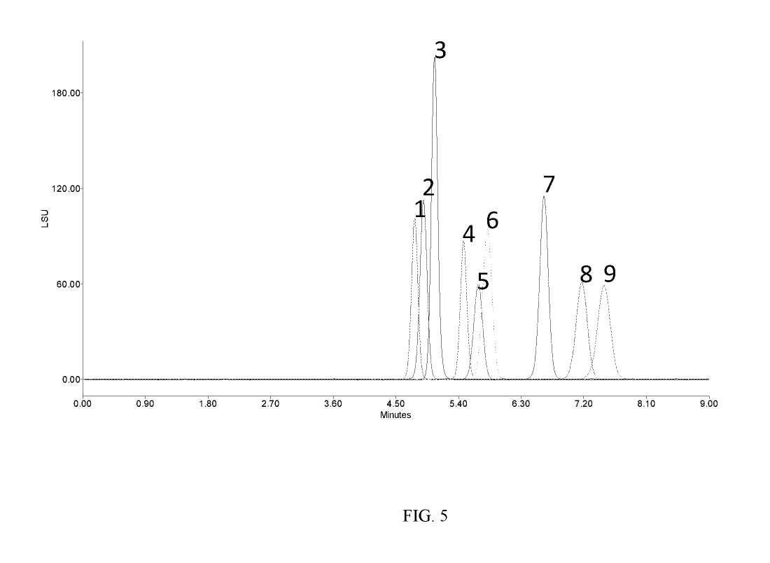

As seen from FIG. 5, the chromatographic conditions show single sharp peak for all compounds. There is no indication of tautomeric resolution.

### Example 6

Each of these stock solutions from Example 1 was injected in an UPLC system with an FLARE HILIC column at a column temperature of 50° C., eluent consisting of acetonitrile and water (92/8) at a flow rate of 0.2 mL/min with ELS detection at a nitrogen pressure of 30 psi and a drift tube temperature of 55° C. Individual injections of each carbohydrate overlaid.

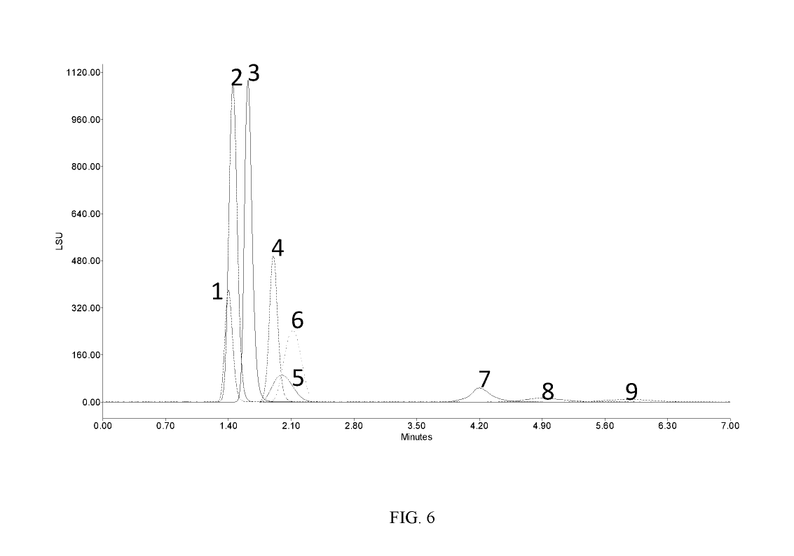

FIG. 6 shows a commercialized column used under exploratory conditions for the analysis of mono-and di-saccharides. The peaks are broad especially for peaks 7-9, but no indication of tautomeric resolution is present.

### Example 7

Each of the stock solutions from Example 1 and the stock solutions of D-(−)-ribose, L-(−)-lyxose, D-allose, D-altrose, L-(+)-gulose, D-psicose, D-(+)-sorbose, D-(−)-tagatose, D-(+)-turanose, and palatinose using the same concentration as Example 1 was injected in an UPLC system with an ACQUITY UPC2™ Torus Diol column at a column temperature of 20° C., eluent consisting of acetonitrile and water (92/8) at a flow rate of 0.2 mL/min with ELS detection at a nitrogen pressure of 30 psi and a drift tube temperature of 55° C.

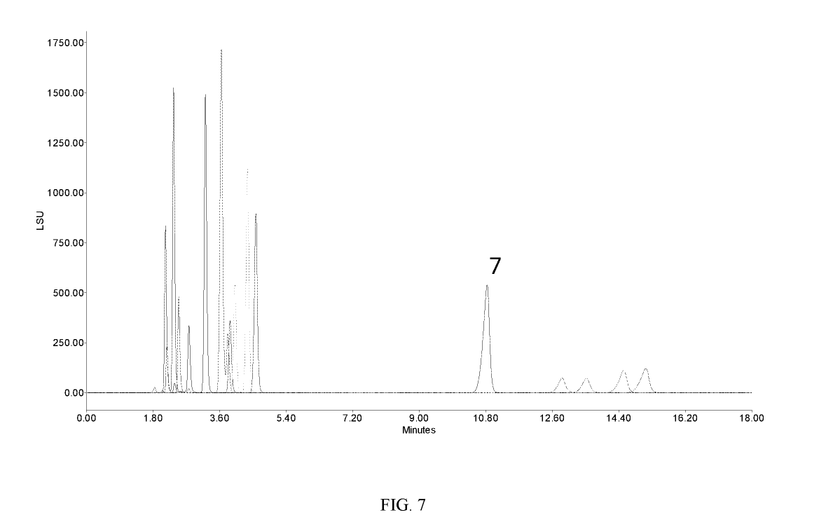

FIG. 7 is overlay of individual injections of each carbohydrate overlaid, and shows multiple peaks demonstrating tautomeric resolution for all reducing mono- and disaccharides. The non-reducing disaccharide, sucrose (peak 7) is present as a single peak.

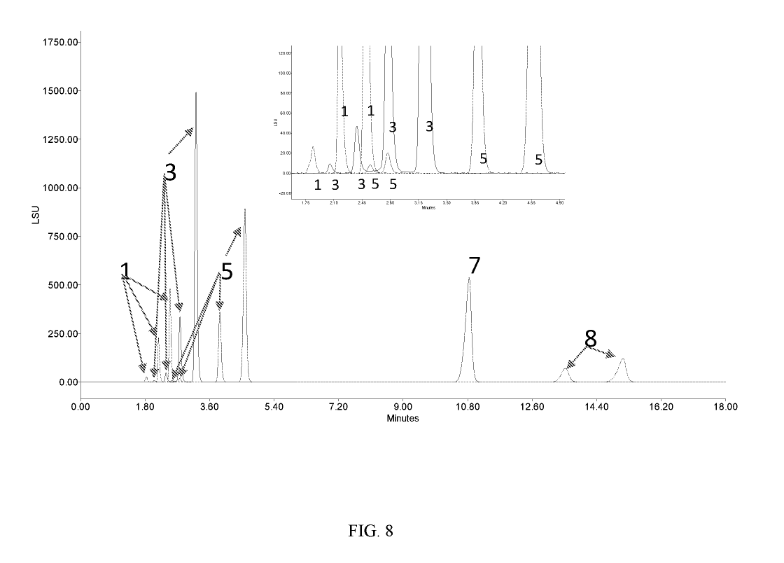

FIG. 8 shows a subset of the mono- and disaccharides (peaks 1, 3, 5 and 8) in relation to the single peak 7 for sucrose. Sharp highly resolved peaks for the tautomeric species are observed.

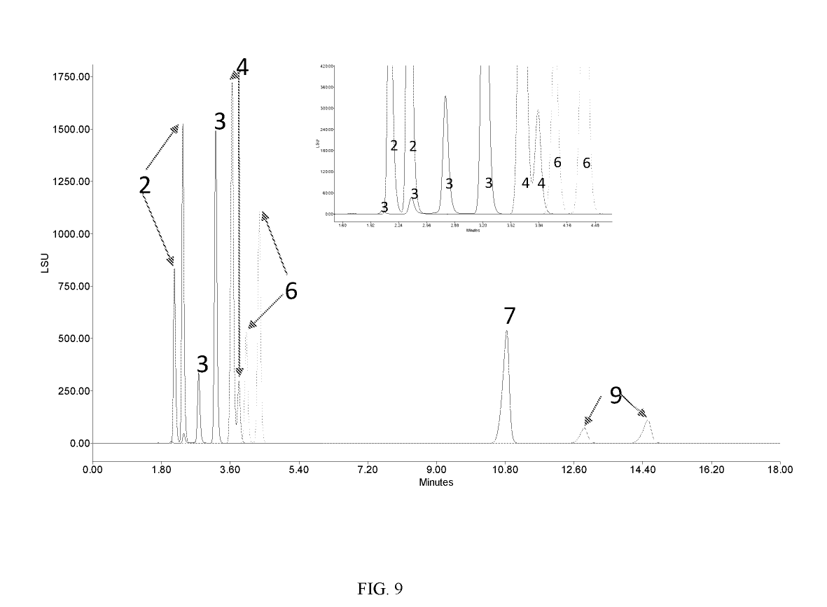

FIG. 9 shows a subset of the mono- and disaccharides (peaks 2, 3, 4, 6 and 9) in relation to the single peak 7 for sucrose. Sharp highly resolved peaks for the tautomeric species are observed.

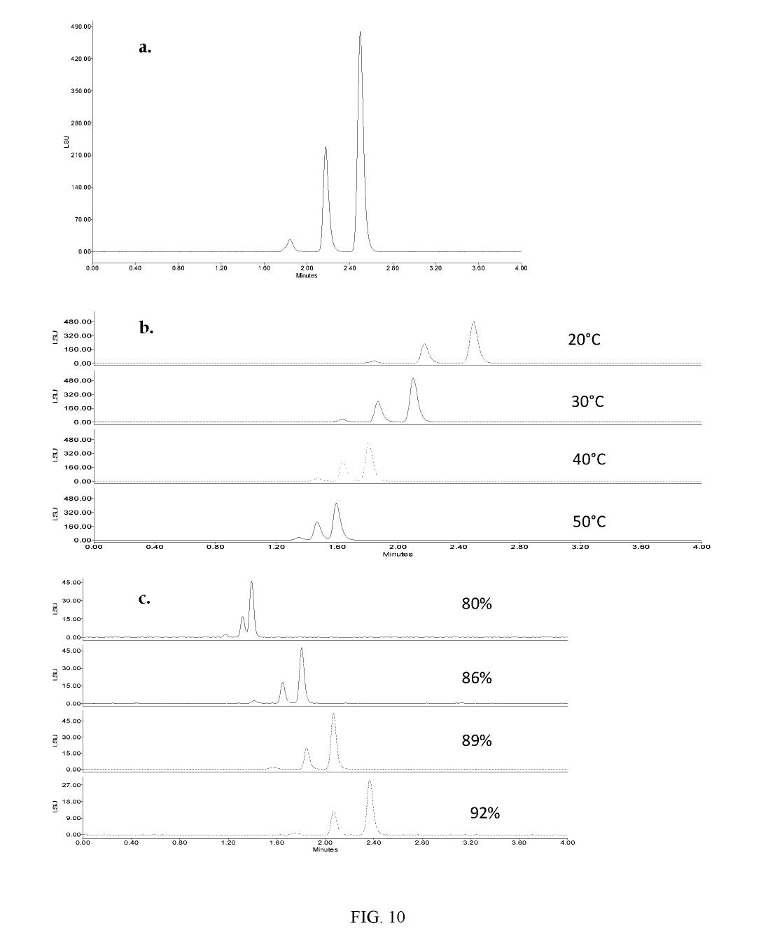

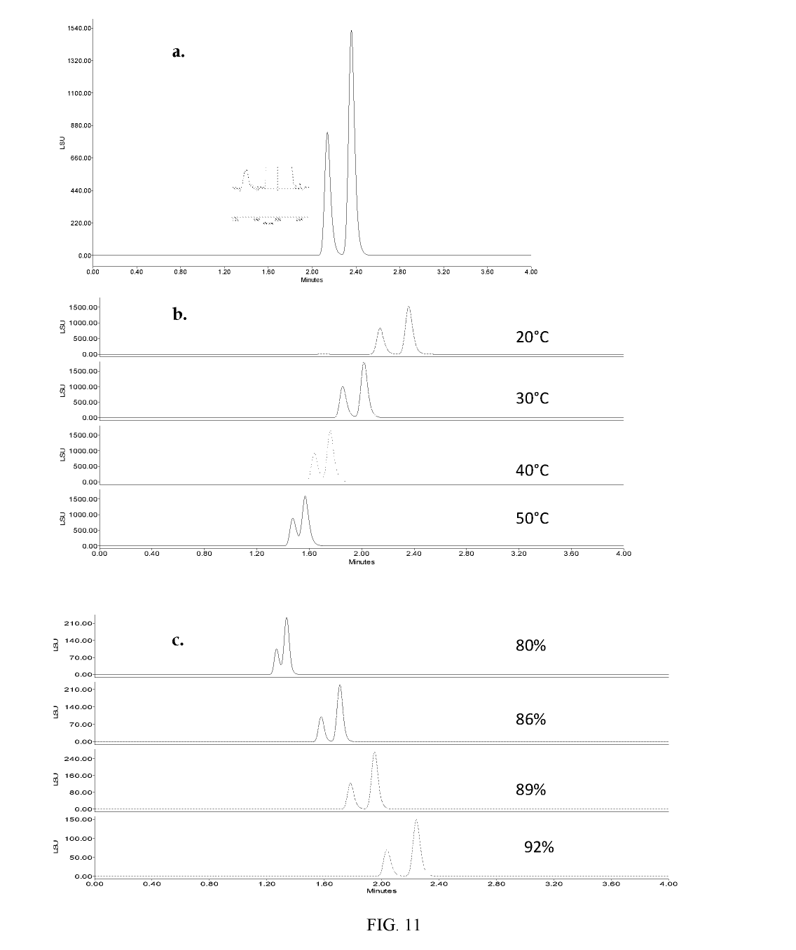

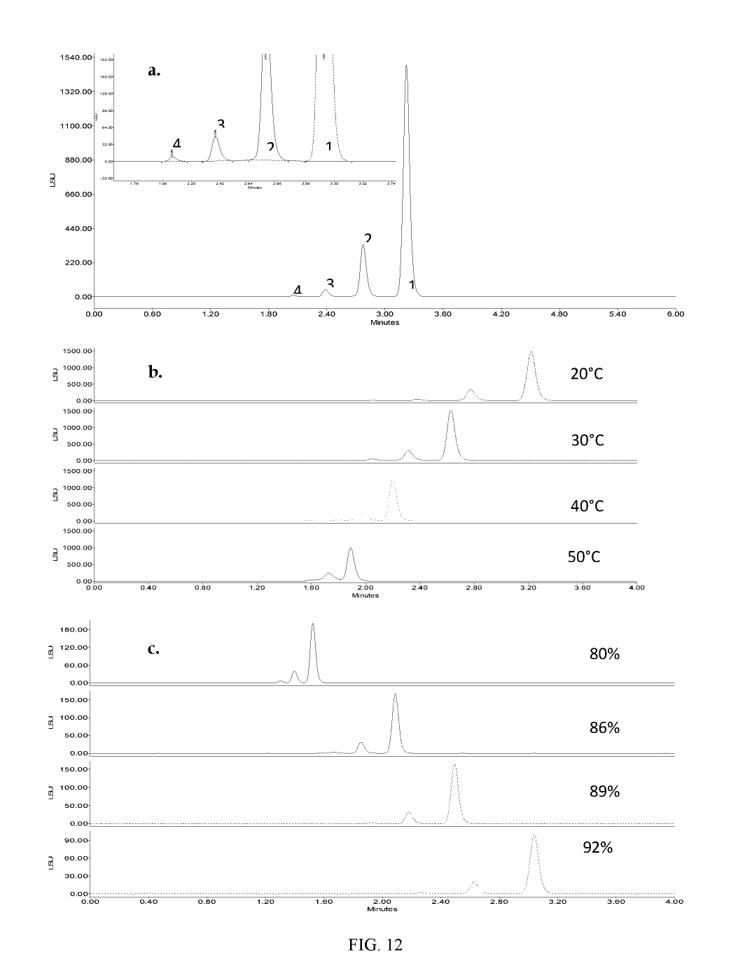

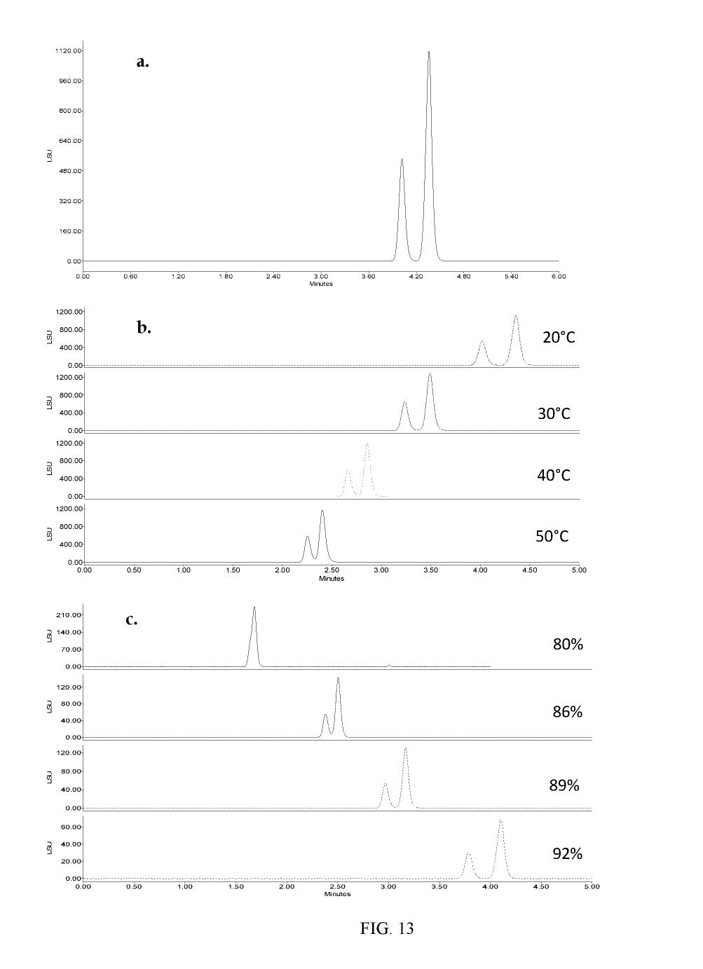

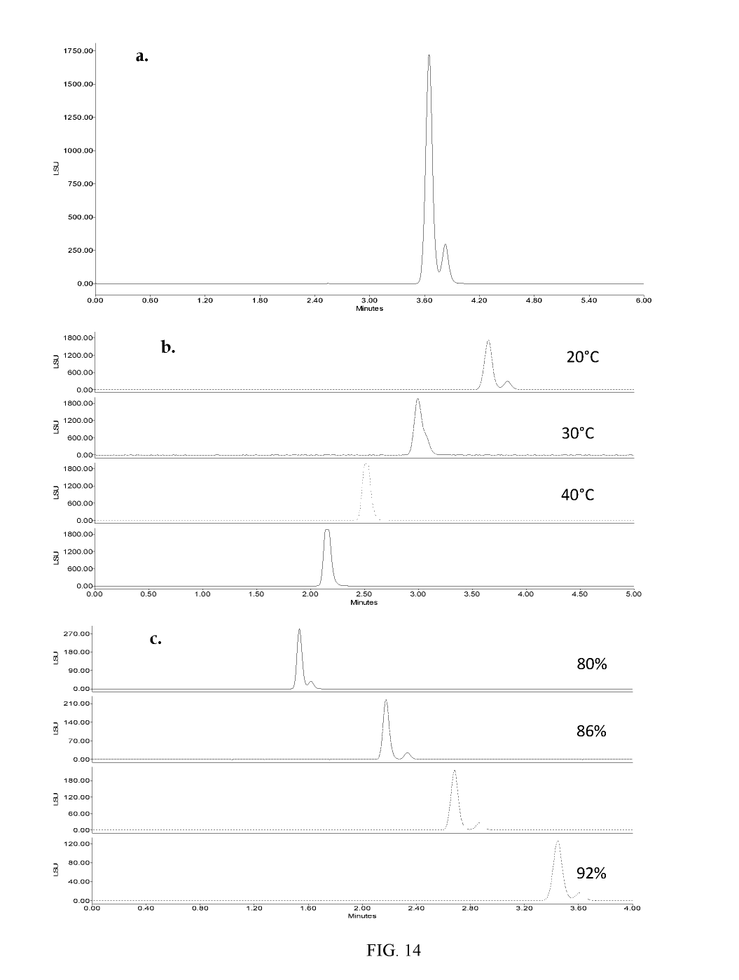

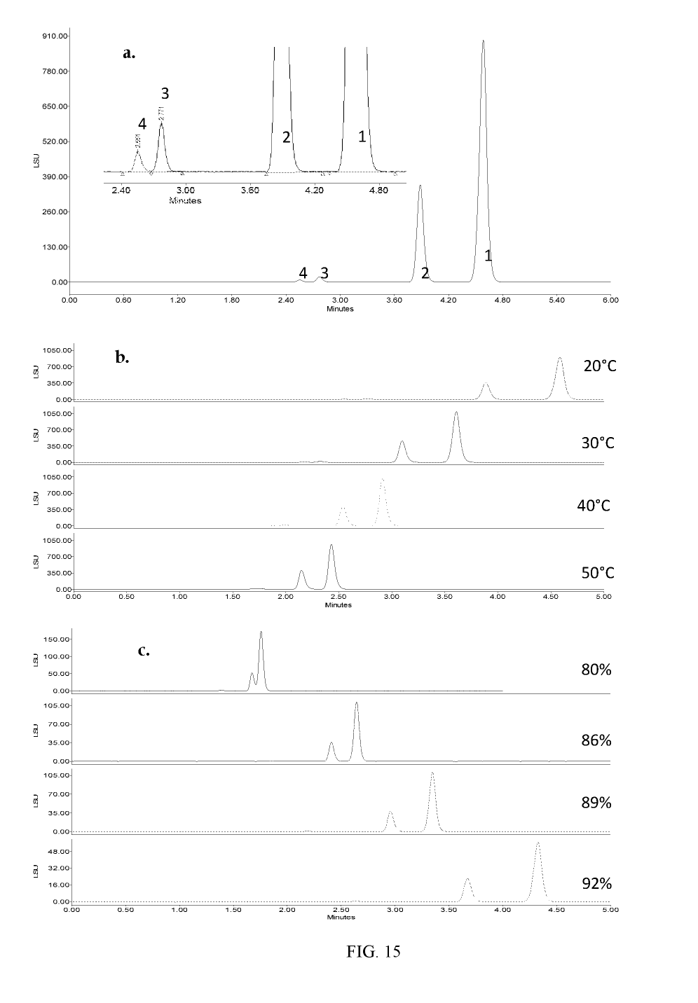

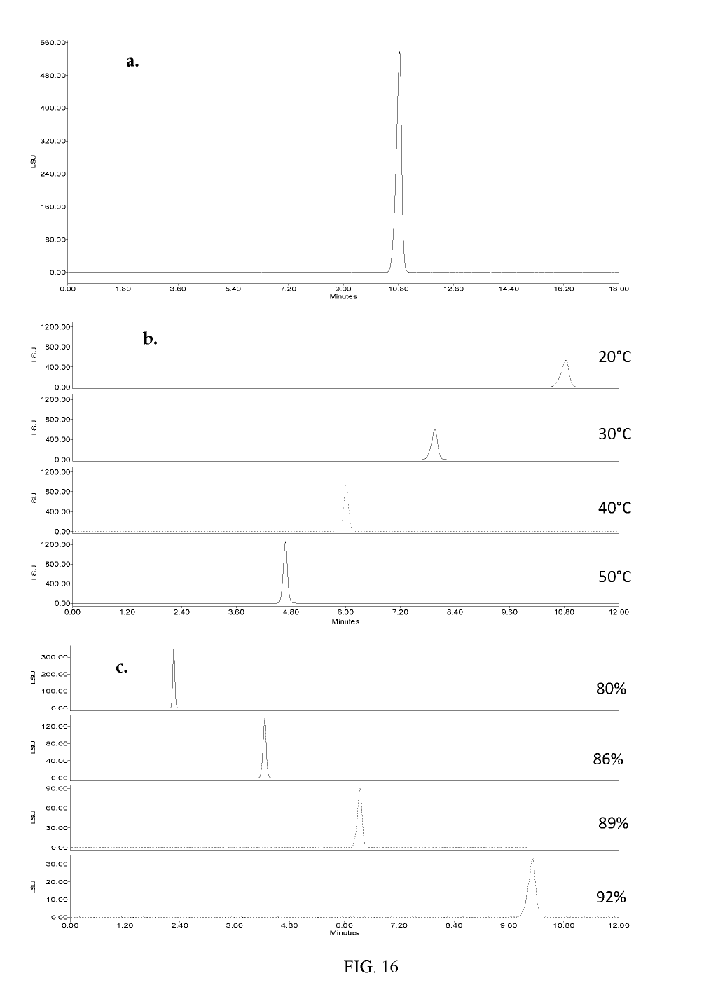

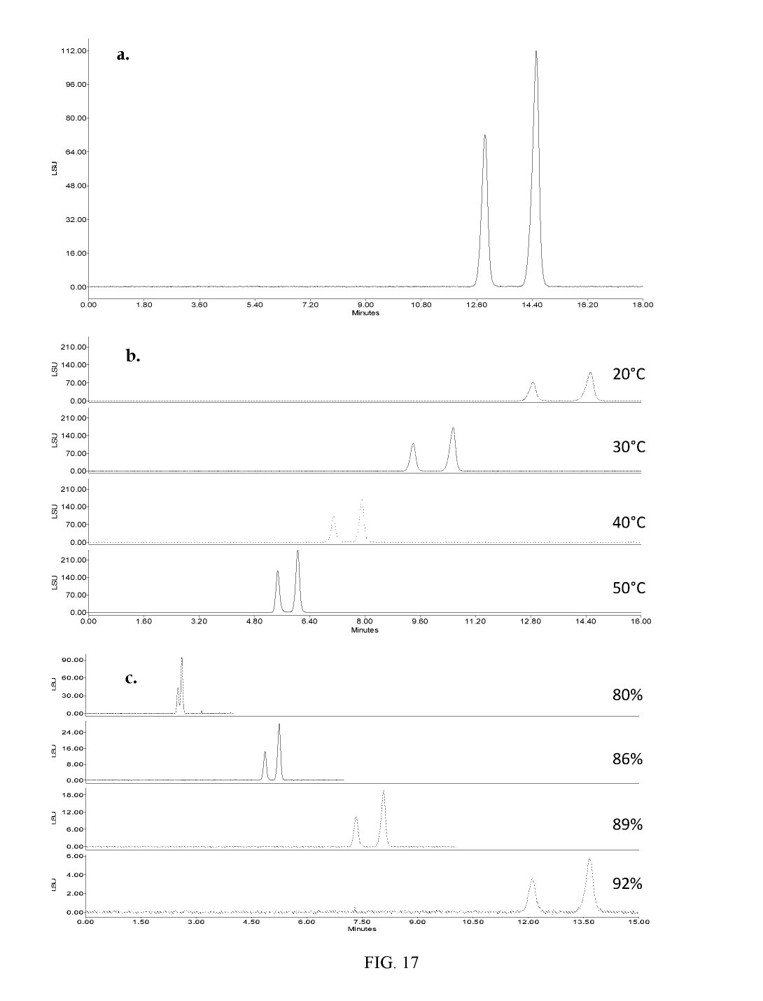

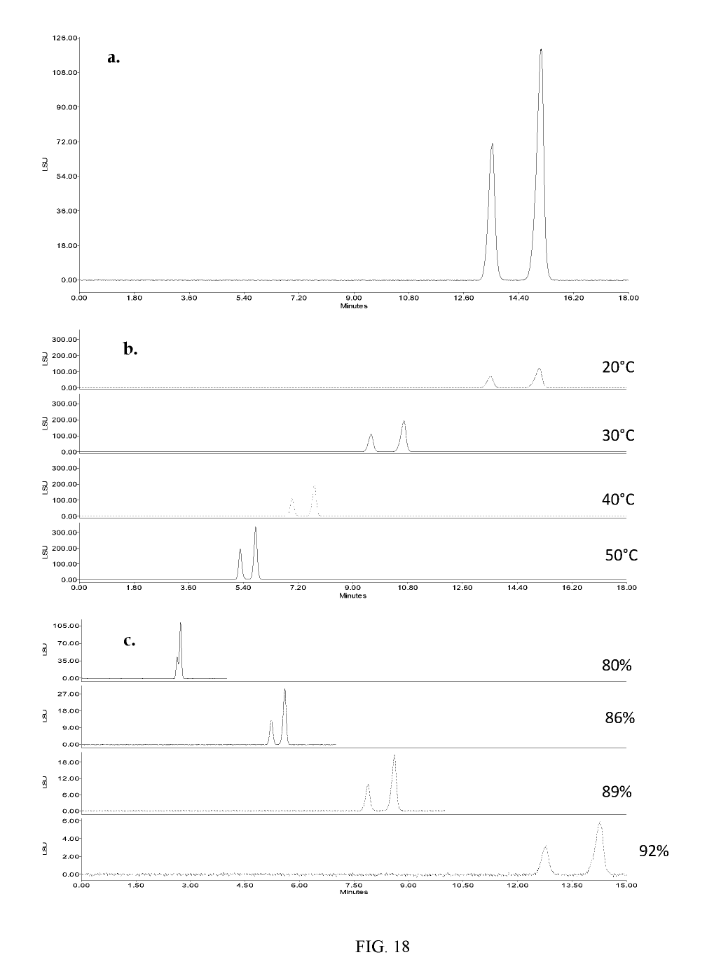

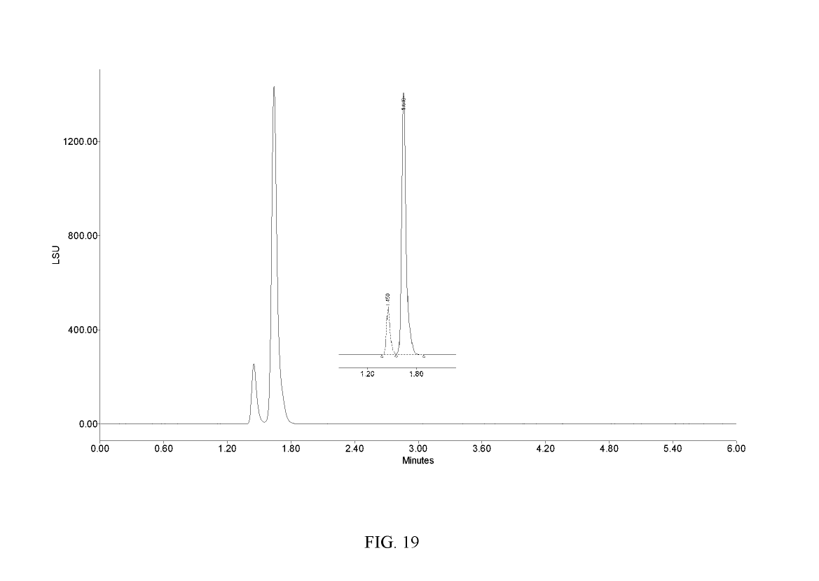

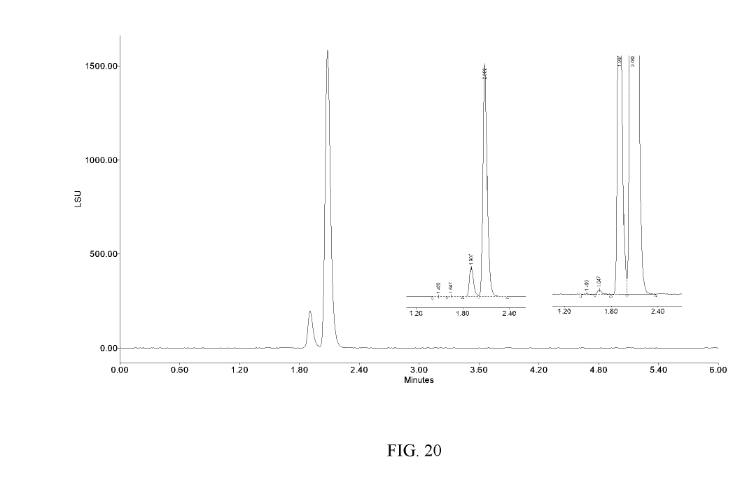

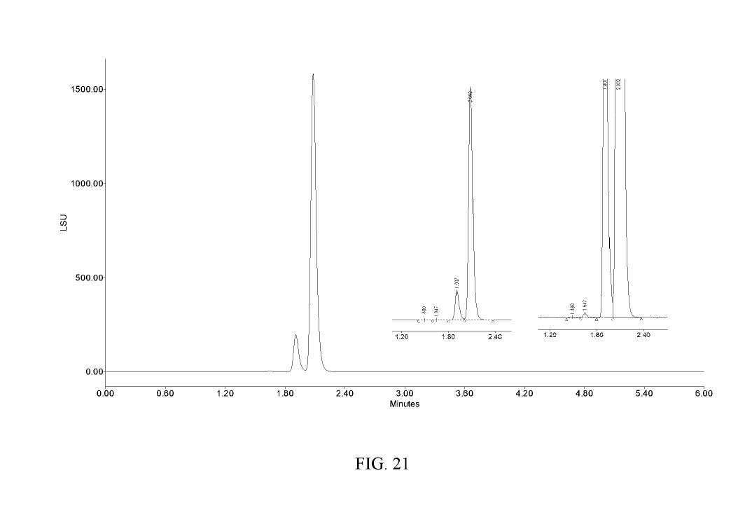

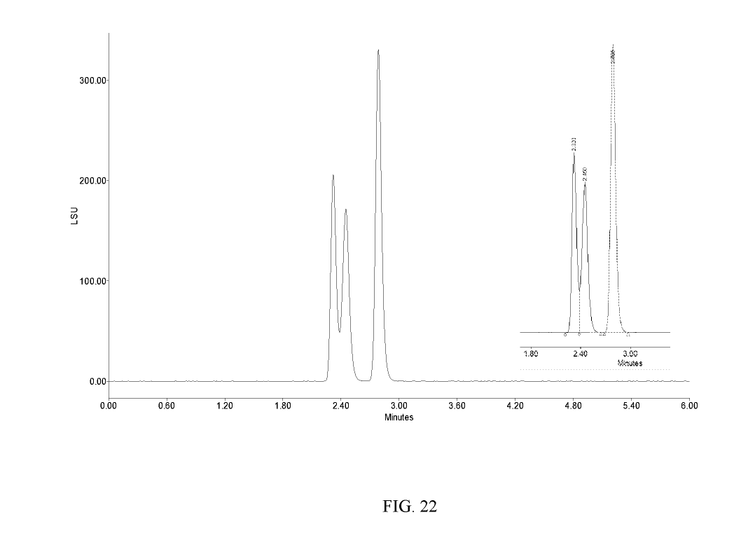

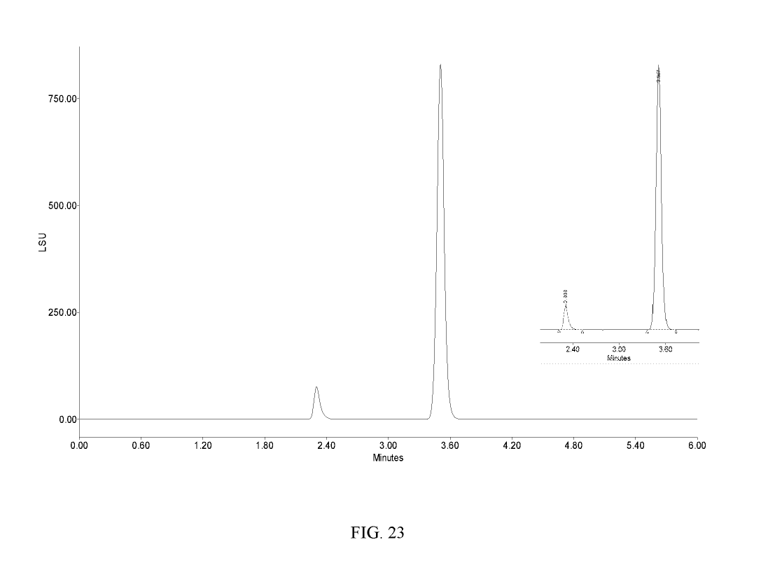

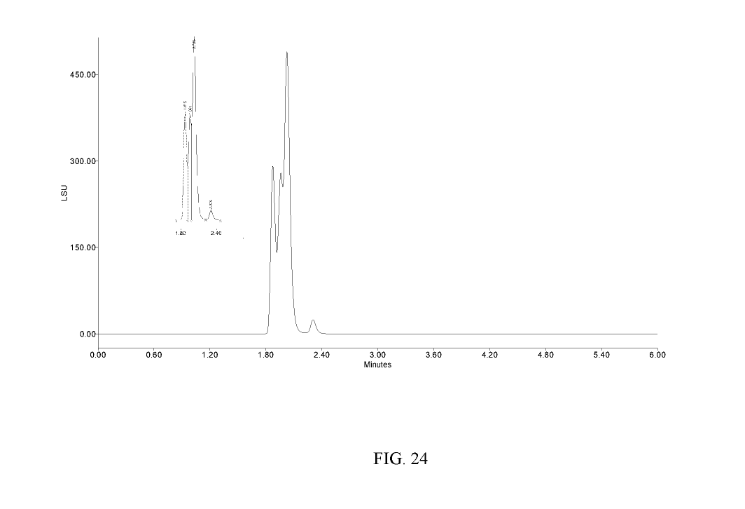

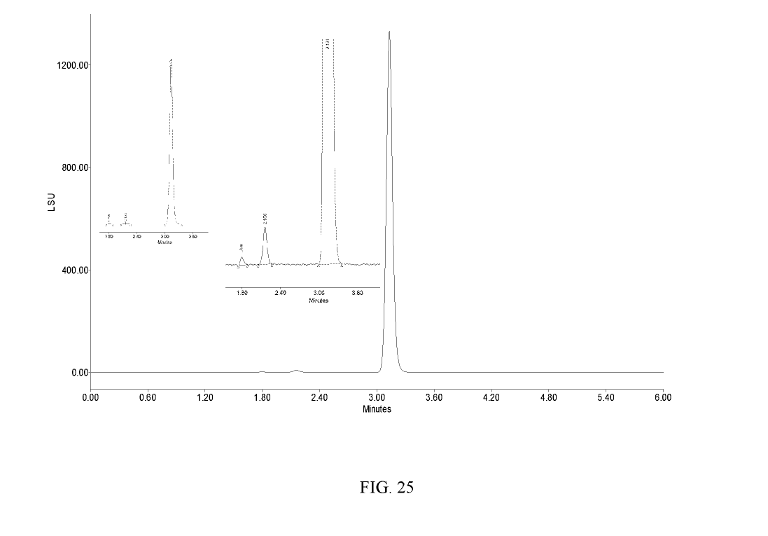

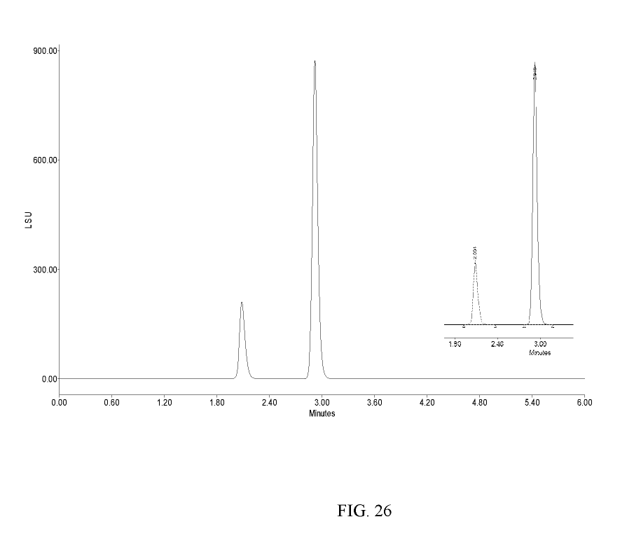

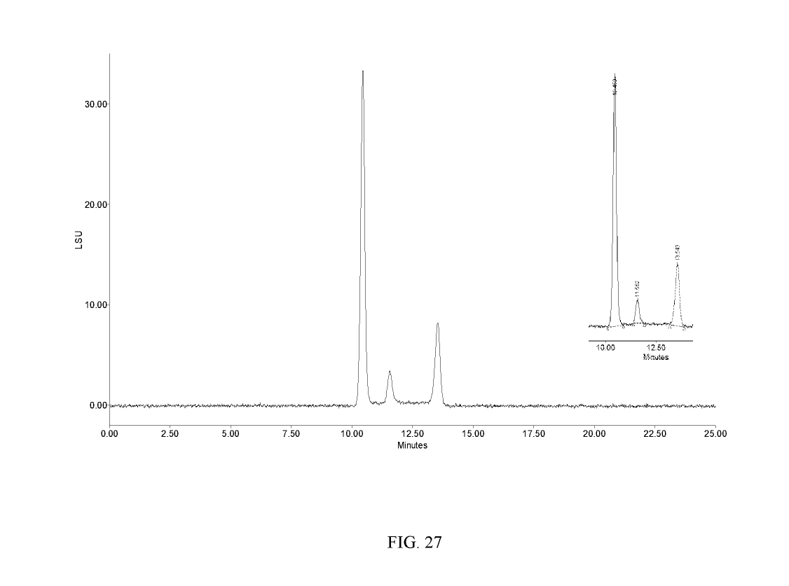

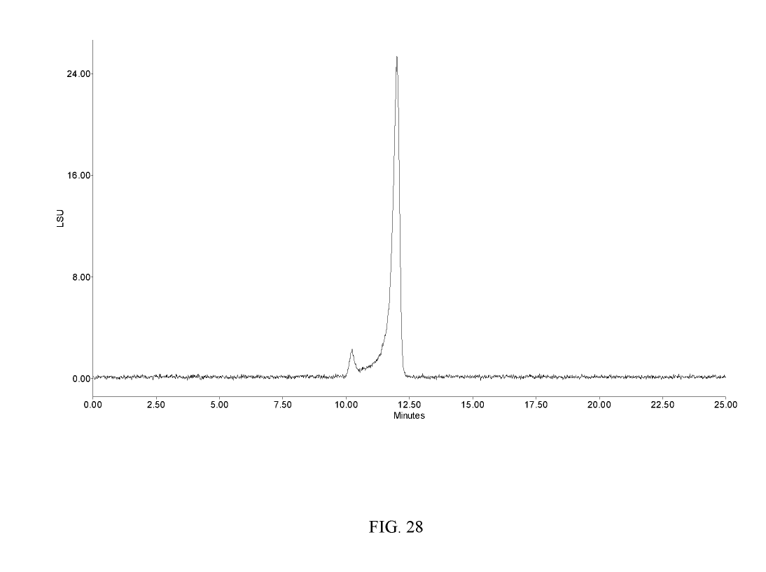

FIGS. 10a-18a, 19-28 show the separation of Arabinose, D-(+)-xylose tautomers, D-(−)-fructose tautomers, D-(+)-glucose tautomers, D-(+)-mannose tautomers, D-(+)-galactose tautomers, Sucrose, D-(+)-maltose (disaccharide) tautomers, Lactose (disaccharide) tautomers, D-(−)-ribose, L-(−)-lyxose, D-allose, D-altrose, L-(+)-gulose, D-psicose, D-(+)-sorbose, D-(−)-tagatose, D-(+)-turanose, and palatinose, respectively.

### Example 8

Each of the stock solutions from Example 1 was injected in an UPLC system with an ACQUITY UPC2™ Torus Diol column at a column temperature of 20° C., 30° C., 40° C., or 50° C., eluent consisting of acetonitrile and water (92/8) at a flow rate of 0.2 mL/min with ELS detection at a nitrogen pressure of 30 psi and a drift tube temperature of 55° C.

FIGS. 10b-18b show the effect of column temperature on the separation of Arabinose, D-(+)-xylose tautomers, D-(−)-fructose tautomers, D-(+)-glucose tautomers, D-(+)-mannose tautomers, D-(+)-galactose tautomers, Sucrose, D-(+)-maltose (disaccharide) tautomers, Lactose (disaccharide) tautomers. The results suggest that the resolution of the tautomers decreases as temperature increases, but in most cases does not fully collapse into a single peak (mannose being the exception).

### Example 9

Each of the stock solutions from Example 1 was injected in an UPLC system with an ACQUITY UPC2™ Torus Diol column at a column temperature of 20° C., eluent consisting of acetonitrile and water of 92/8, 89/11, 86/14, or 80/20, at a flow rate of 0.2 mL/min with ELS detection at a nitrogen pressure of 30 psi and a drift tube temperature of 55° C.

FIGS. 10c-18c show the effect of acetonitrile percentage in the mobile phase on the separation of Arabinose, D-(+)-xylose tautomers, D-(−)-fructose tautomers, D-(+)-glucose tautomers, D-(+)-mannose tautomers, D-(+)-galactose tautomers, Sucrose, D-(+)-maltose (disaccharide) tautomers, Lactose (disaccharide) tautomers. The results suggest that as the concentration of acetonitrile decreases, the retention times decreases and the resolution of the tautomers decreases.

### Example 10

α-D-Glucose (19.8 mg) was dissolved in a volumetric flask, and brought to the mark with Milli-Q water. The solution was immediately placed into a 1.2 mL vial and the first injection of 0.4 μL was started immediately. After the first injection, injection were made every 5 minutes for a total of 40 injections.

Each of the sample was injected in an UPLC system with an ACQUITY UPC2™ Torus Diol column at a column temperature of 20° C., eluent consisting of acetonitrile and water (92/8) at a flow rate of 0.2 mL/min with ELS detection at a nitrogen pressure of 30 psi and a drift tube temperature of 55° C.

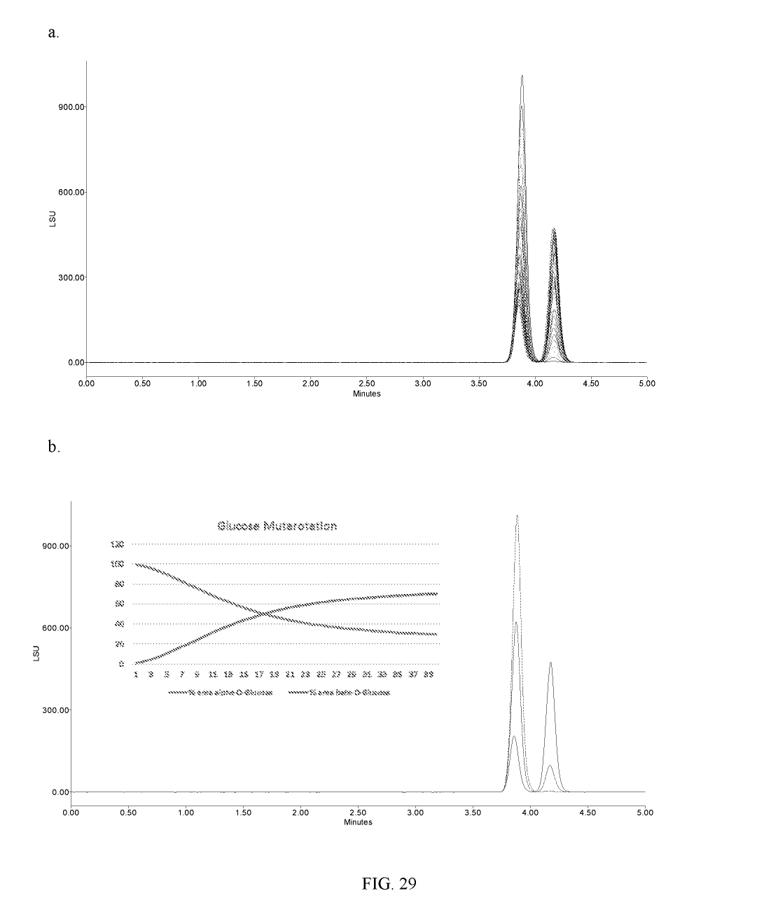

FIGS. 29a and 29b illustrates the mutarotation of α-D-(+)-glucose tautomer in water. As the storage time of α-D-(+)-glucose tautomer in water increases, the amount of α-D-(+)-glucose tautomer decreases and the amount of β-D-(+)-glucose tautomer increases.

### Example 11

Each of the stock solutions from Example 1 was injected in an UPLC system with an ACQUITY UPLC Glycan BEH Amide column (50 mm×2.1 mm i.d., particle size 1.7 μm) at a column temperature of 20° C., eluent consisting of acetonitrile and water (86/14) at a flow rate of 0.2 mL/min with ELS detection at a nitrogen pressure of 30 psi and a drift tube temperature of 55 degrees C.

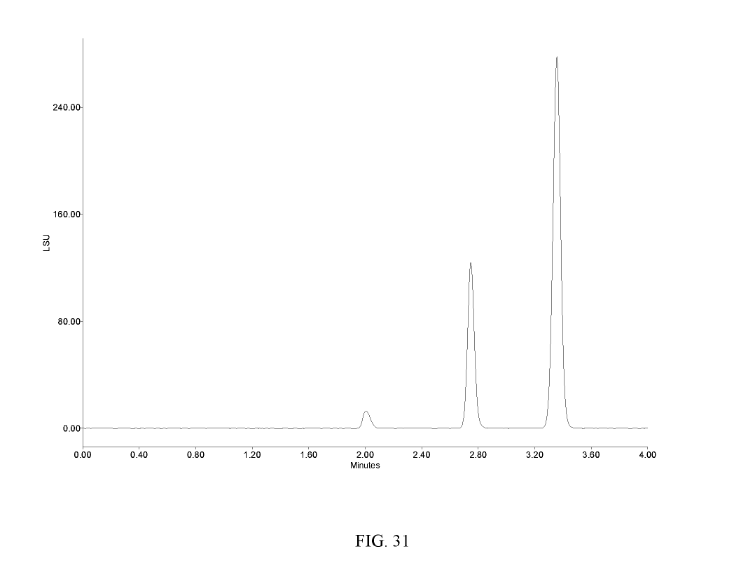

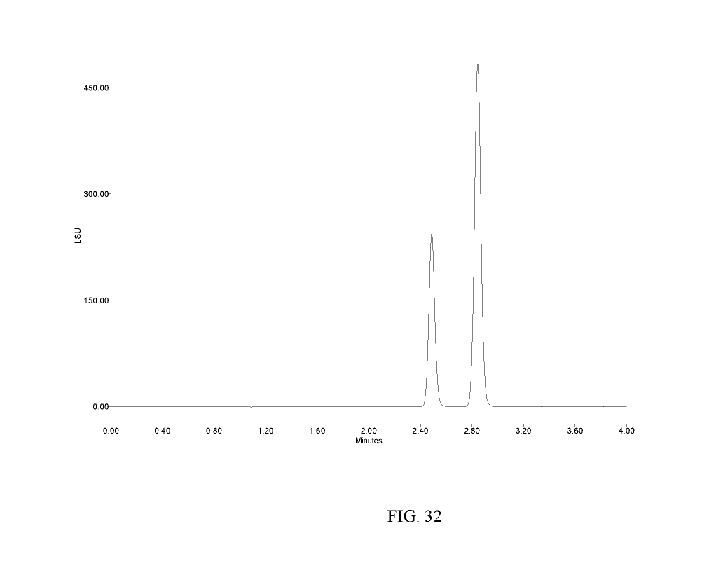

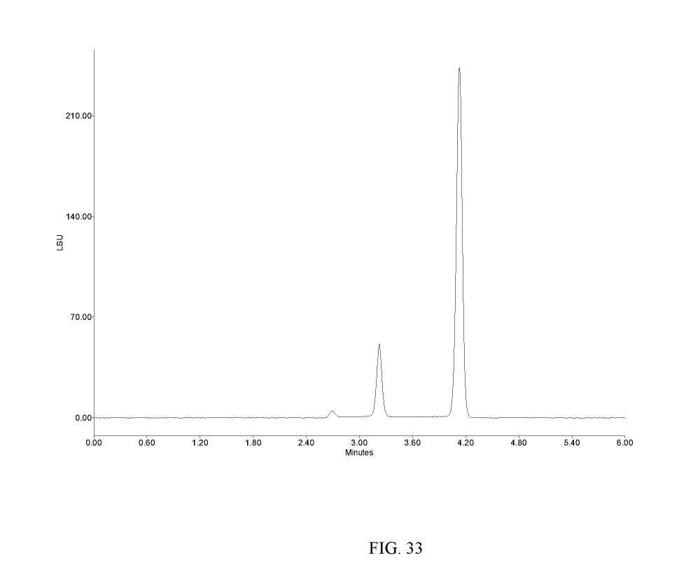

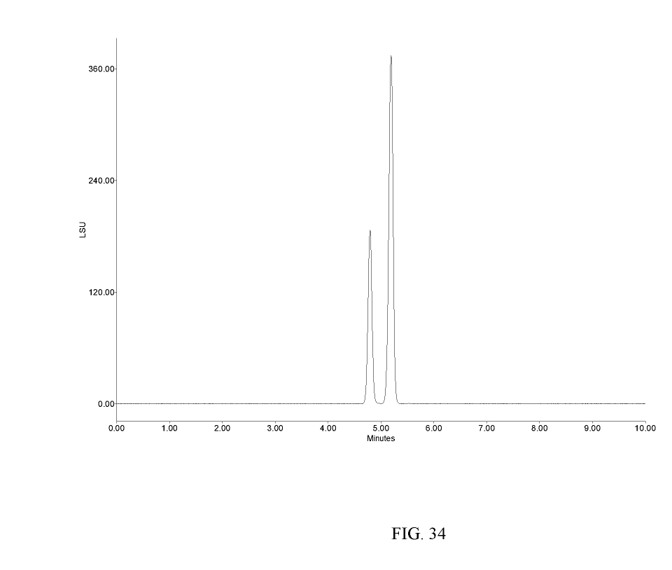

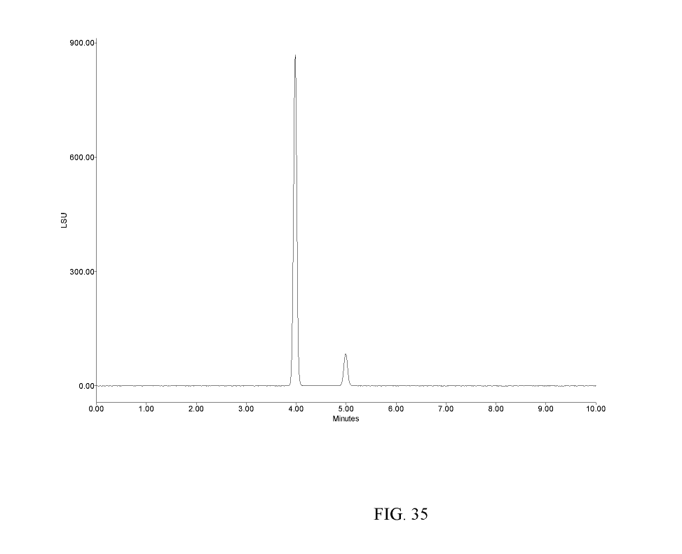

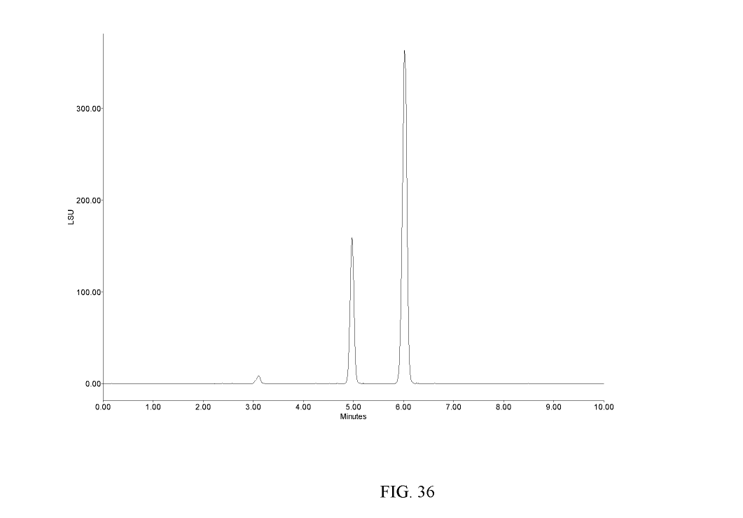

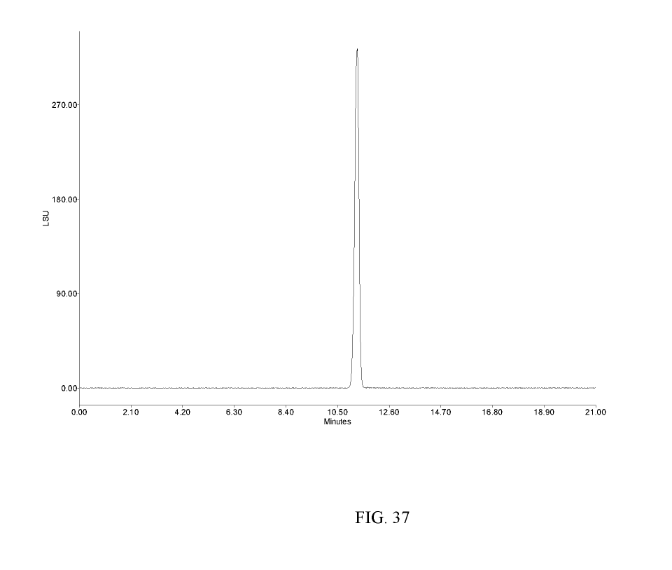

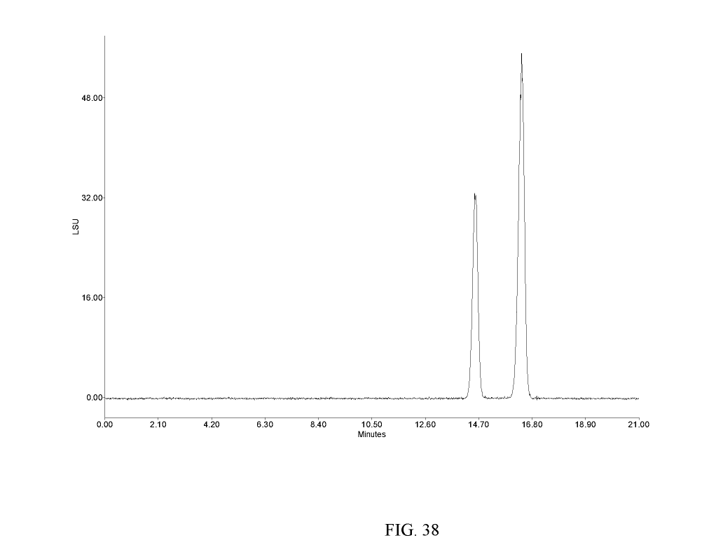

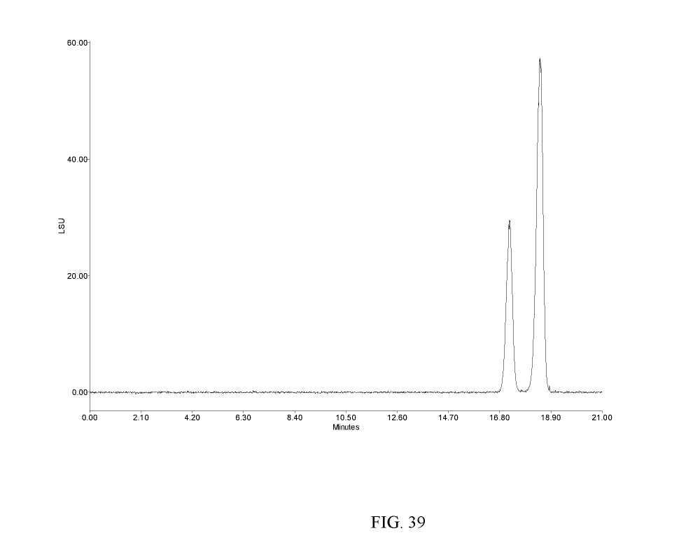

FIGS. 31-39 show the separation of Arabinose, D-(+)-xylose tautomers, D-(−)-fructose tautomers, D-(+)-glucose tautomers, D-(+)-mannose tautomers, D-(+)-galactose tautomers, Sucrose, D-(+)-maltose (disaccharide) tautomers, and lactose (disaccharide) tautomers, respectively. This is a second column that is capable of providing the resolution of tautomeric species of mono- and di-saccharides.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

1. A method of separating carbohydrate tautomeric species comprising:
chromatographically separating tautomeric species using a chromatographic device;
wherein the chromatographic device comprising
a column packed with chromatographic stationary phase represented by Formula 1 or 2,
[X](W)a(Q)b(T)c Formula 1
wherein:
X is a high purity chromatographic core composition having a surface comprising
a silica core material, metal oxide core material, an inorganic-organic hybrid material or a
group of block copolymers thereof;
W is absent and/or includes hydrogen and/or includes hydroxyl on the surface of X;
Q is a functional group that minimizes retention variation over time (drift) under chromatographic conditions utilizing low water concentrations;
T comprises one or more hydrophilic, polar, ionizable, and/or charged functional groups that chromatographically interact with the analyte; and
b and c are positive numbers, 0.05<(b/c)<100, and a>0.

2. The method of separating carbohydrate tautomeric species according to claim 1 further comprising an evaporative light scattering (ELS) detector, or a refractive index (RI) detector.

3. The method of separating carbohydrate tautomeric species according to claim 1, wherein the chromatographically separating tautomeric species uses acetonitrile and water as mobile phases.

4. The method of separating carbohydrate tautomeric species according to claim 1, wherein the chromatographically separating tautomeric species uses acetone and water as mobile phases.

5. The method of separating carbohydrate tautomeric species according to claim 1, wherein the chromatographic device is connected to a detector selected from the group consisting of mass spectrometers, charged aerosol, polarimeter, pulsed amperometric electrochemical, condensation nucleation light scattering, nuclear magnetic resonance (nmr), or combinations thereof.

6. The method of separating carbohydrate tautomeric species according to claim 1, wherein the tautomeric species are obtained from mono- and disaccharides and their mixtures.

7. The method of separating carbohydrate tautomeric species according to claim 1, wherein the tautomeric species are arabinose, xylose, fructose, mannose, galactose, sucrose, glucose, lactose, maltose, sorbose, ribose, psicose, trehalose, raffinose, altrose, tagatose, lyxose, turanose, allose, gulose, palatinose, or mixtures thereof.

8. The method of separating carbohydrate tautomeric species according to claim 1, wherein the tautomeric species are arabinose tautomers.

9. The method of separating carbohydrate tautomeric species according to claim 1, wherein the tautomeric species are xylose tautomers.

10. The method of separating carbohydrate tautomeric species according to claim 1, wherein the tautomeric species are fructose tautomers.

11. The method of separating carbohydrate tautomeric species according to claim 1, wherein the tautomeric species are mannose tautomers.

12. The method of separating carbohydrate tautomeric species according to claim 1, wherein the tautomeric species are galactose tautomers.

13. The method of separating carbohydrate tautomeric species according to claim 1, wherein the tautomeric species are glucose tautomers.

14. The method of separating carbohydrate tautomeric species according to claim 1, wherein the tautomeric species are lactose tautomers.

15. The method of separating carbohydrate tautomeric species according to claim 1, wherein the tautomeric species are maltose tautomers.

16. A method of separating carbohydrate tautomeric species comprising:
chromatographically separating a sample using a chromatographic device;
wherein the chromatographic device comprising
a column packed with chromatographic stationary phase represented by Formula 1
[X](W)a(Q)b(T)c Formula 1
wherein:
X is a chromatographic core material comprising a silica based, metal oxide based, or inorganic-organic hybrid based core surface;
W is absent and/or includes hydrogen and/or includes hydroxyl on the surface of X;
Q comprises one or more functional groups that essentially prevent chromatographic interaction between an analyte, and X and W, wherein a first fraction of Q is bound to X and a section fraction of Q is polymerized; T comprises one or more hydrophilic, polar, ionizable, and/or charged functional groups that chromatographically interact with the analyte, wherein a third fraction of T is bound to Q, a fourth fraction of T is bound to X, and a fourth fraction of T is polymerized; and
b and c are positive numbers, 0.05<(b/c)<100, and a>0.

17. The method of separating carbohydrate tautomeric species according to claim 16 further comprising an evaporative light scattering (ELS) detector, or a refractive index (RI) detector.

18. The method of separating carbohydrate tautomeric species according to claim 16, wherein the chromatographically separating tautomeric species uses acetonitrile and water as mobile phases.

19. The method of separating carbohydrate tautomeric species according to claim 1, wherein the chromatographically separating tautomeric species uses acetone and water as mobile phases.

20. The method of separating carbohydrate tautomeric species according to claim 16, wherein the chromatographic device is connected to a detector selected from the group consisting of mass spectrometers, charged aerosol, polarimeter, pulsed amperometric electrochemical, nuclear magnetic resonance (nmr), or combinations thereof.

21. The method of separating carbohydrate tautomeric species according to claim 16, wherein the tautomeric species are mono- and disaccharides tautomeric species and their mixtures.

22. The method of separating carbohydrate tautomeric species according to claim 16, wherein the tautomeric species are arabinose, xylose, fructose, mannose, galactose, sucrose, glucose, lactose, maltose, sorbose, ribose, psicose, trehalose, raffinose, altrose, tagatose, lyxose, turanose, allose, gulose, palatinose, or mixtures thereof.

23. The method of separating carbohydrate tautomeric species according to claim 16, wherein the tautomeric species are arabinose tautomers.

24. The method of separating carbohydrate tautomeric species according to claim 16, wherein the tautomeric species are xylose tautomers.

25. The method of separating carbohydrate tautomeric species according to claim 16, wherein the tautomeric species are fructose tautomers.

26. The method of separating carbohydrate tautomeric species according to claim 16, wherein the tautomeric species are mannose tautomers.

27. The method of separating carbohydrate tautomeric species according to claim 16, wherein the tautomeric species are galactose tautomers.

28. The method of separating carbohydrate tautomeric species according to claim 16, wherein the tautomeric species are glucose tautomers.

29. The method of separating carbohydrate tautomeric species according to claim 16, wherein the tautomeric species are lactose tautomers.

30. The method of separating carbohydrate tautomeric species according to claim 16, wherein the tautomeric species are maltose tautomers.

31. A method of separating carbohydrate tautomeric species comprising:
chromatographically separating tautomeric species using a chromatographic device;
wherein the chromatographic device comprising
a column packed with a copolymer comprising at least one hydrophilic monomer and a poly-amide bonded phase, wherein the average pore diameter is greater than or equal to about 200 Å.
